SEQUENCE LISTING (4 sequences)

SEQ ID NO: 1 - protein, 16 aa, Homo sapiens
VITAIAKFERLQTVTN

SEQ ID NO: 2 - protein, 16 aa, Homo sapiens
VITAIAKFERLQTVTN

SEQ ID NO: 3 - protein, 16 aa, Homo sapiens
TAIAKFERLQTVTNYF

SEQ ID NO: 4 - protein, 16 aa, Homo sapiens
LVITAIAKFERLQTVT

(19) United States
(12) Patent Application Publication
(10) Pub. No.: US 2018/0353567 A1
(43) Pub. Date: Dec. 13, 2018

(54) TREATMENT OF CARDIOVASCULAR DISEASE WITH COMPOUNDS THAT PROMOTE SELECTIVE INTERACTION OF THE B2-ADRENERGIC RECEPTOR WITH B-ARRESTIN

(71) Applicants: THOMAS JEFFERSON UNIVERSITY, PHILADELPHIA, PA (US); TEMPLE UNIVERSITY - OF THE COMMONWEALTH SYSTEM OF HIGHER EDUCATION, Philadelphia, PA (US)

(72) Inventors: Jeffrey L. Benovic, Philadelphia, PA (US); Richard Carr, III, Denver, PA (US); Douglas G. Tilley, Bryn Mawr, PA (US)

(73) Assignee: THOMAS JEFFERSON UNIVERSITY, PHILADELPHIA, PA (US)

(21) Appl. No.: 15/768,428

(22) PCT Filed: Oct. 17, 2016
(86) PCT No.: PCT/US16/57338

§ 371 Date: Apr. 13, 2018

Related U.S. Application Data
(60) Provisional application No. 62/242,152, filed on Oct. 15, 2015.

(51) Int. Cl. A61K 38/10; A61P 9/00; A61K 9/00; A61K 9/127; A61K 31/138

(52) U.S. Cl. CPC A61K 38/10; A61P 9/00; A61K 31/138; A61K 9/127; A61K 9/0019

(57) ABSTRACT

A pharmaceutical composition and methods of administering the same for treatment of cardiovascular disease comprising a pepducin having a sequence SEQ ID No. 1, wherein said composition stimulates cardiomyocyte contractility and activating the β2AR/β-arrestin signaling pathway.

### PRIORITY CLAIM

This application claims the benefit of U.S. Provisional Application Ser. No. 62/242,152 filed Oct. 15, 2015, the disclosure contents of which are hereby incorporated by reference in their entirety.

### GOVERNMENT SUPPORT CLAUSE

This invention was made with government support under GM047417, HL114471, GM100836 and HL105414 awarded by the National Institutes of Health. The government has certain rights in the invention.

### SEQUENCE LISTING

The instant application contains a Sequence Listing which has been submitted in ST. 25 Text File Format via EFS-WEB with the United States Receiving Office and is hereby incorporated by reference in its entirety.

### FIELD OF INVENTION

The present application is generally related to methods of treating cardiovascular diseases with compounds that promote selective interaction of the β2-adrenergic receptor with β-arrestin.

### BACKGROUND OF INVENTION

β-adrenergic receptors (βARs) are critical regulators of acute cardiovascular physiology. In response to elevated catecholamine stimulation during the development of congestive heart failure (CHF), chronic activation of Gs-dependent β1AR and Gi-dependent β2AR pathways leads to enhanced cardiomyocyte death, reduced β1AR expression and decreased inotropic reserve. β-blockers act to block excessive catecholamine stimulation of βARs to decrease cellular apoptotic signaling and normalize β1AR expression and inotropy. While these actions reduce cardiac remodeling and mortality outcomes, the effects are not sustained.

Commonly prescribed drugs for congestive heart failure (CHF) include β-adrenergic receptor antagonists or β-blockers. These drugs operate by inhibiting deleterious apoptotic signaling and normalizing inotropic signaling from these receptors. As the β1AR (dominant subtype in the heart) is systematically downregulated during CHF while Gi (a G protein that antagonizes contractile signaling) is upregulated, the ability to selectively control β2AR signaling becomes an attractive therapeutic approach. Further, it is proposed that biasing β2AR interaction with β-arrestins versus G proteins may be therapeutically advantageous for the treatment of CHF because β-arrestins have been shown to promote anti-apoptotic signaling and may increase cardiomyocyte contractility through mechanisms that are distinct from those engaged by G proteins.

Carvedilol, a currently prescribed non-selective β-blocker for treating heart failure, has been classified as a β-arrestin-biased agonist that can inhibit basal signaling from βARs and also stimulate cell survival signaling pathways. Other β-antagonists such as bisoprolol and metoprolol are β1AR selective β-blockers used to treat heart failure and have no β-arrestin-biased activity. However, described herein embodiments are disclosed towards a β-arrestin-biased pepducin of the β2AR that is able to induce cardiomyocyte contractility and anti-apoptotic signaling to provide a pharmacological template for next-generation cardiovascular pharmaceuticals.

### SUMMARY OF INVENTION

A preferred embodiment is directed to a method of treatment of acute HF (AHF) comprising administering to a patient an effective amount of ICL1-9 to enhance cardiac contractility.

A further embodiment is directed to a method for treating congestive heart failure (CHF) comprising administering to a patient an effective amount of ICL1-9 to enhance cardiac contractility and prevent myocardial remodeling.

A further embodiment is directed to a method for treating myocardial infarction and/or ischemia/reperfusion injury comprising administering to a patient an effective amount of ICL1-9 to promote cardiomyocyte survival and contractility.

A further embodiment is directed to a method for treating cardiovascular diseases by administering to a patient a pharmaceutical composition comprising ICL1-9 wherein, the use of ICL1-9 pepducin in a pharmaceutical composition is expected to provide the following benefits to a patient suffering an acute cardiac injury (i.e. myocardial infarction and/or ischemia/reperfusion) or in the context of either acute or chronic HF: (1) decreased catecholamine-induced Gs protein-dependent cardiotoxicity to decrease cell death; (2) active engagement of β-arrestin-dependent survival signaling to promote cell survival, thereby decreasing cell death-induced detrimental myocardial remodeling; and (3) increased cardiomyocyte contractility to actively improve cardiac function.

The formulations as described herein can then be suitably administered to a patient in need thereof to effectuate a method of treating of heart disease, myocardial infarction, ischemia, reperfusion injury, AHF, CHF, and other acute and chronic diseases. Preferred compositions comprise ICL1-9 having SEQ ID No. 1. However, suitable compositions comprising 90% homology with that as defined by SEQ ID No. 1.

A further embodiment is directed to a method of treatment of acute heart failure (AHF) comprising administering to a patient an effective amount of a pepducin having SEQ ID No. 1, which binds to β2AR and stimulates cardiac contractility. A further embodiment is directed towards a method for treating congestive heart failure (CHF) comprising administering to a patient an effective amount of a pepducin having SEQ ID No. 1, which binds to β2AR and stimulates cardiac contractility and prevent myocardial remodeling.

A further embodiment is directed to a method for treating myocardial infarction and/or ischemia/reperfusion injury comprising administering to a patient an effective amount of a pepducin having SEQ ID No. 1, which binds to β2AR, stimulates cardiomyocyte function, and promotes cardiomyocyte survival and contractility.

A further embodiment is directed towards a method of treating cardiovascular diseases by administering to a patient a pharmaceutical composition comprising a pepducin having SEQ ID No. 1 having a binding affinity for β2AR. Further embodiments comprise wherein the pharmaceutical composition stimulates cardioprotective signaling and inotropic effects through the β2AR. Further embodiments comprise wherein the pharmaceutical composition induces cardiomyocyte contraction. Yet further embodiments comprise wherein the pharmaceutical composition enhances contractile function.

A pharmaceutical composition for treating cardiovascular diseases comprising a pepducin having SEQ ID No. 1, wherein said pharmaceutical composition operates independently of the orthosteric ligand binding pocket to stimulate a signaling pathway that promotes contraction of the heart, and wherein said pharmaceutical composition stabilizes a β2AR conformation that is both a substrate for GRK-mediated phosphorylation and β-arrestin binding.

A method for treating cardiovascular diseases by administering to a patient a pharmaceutical composition comprising a pepducin having SEQ ID No. 1 wherein, the use of ICL1-9 pepducin in a pharmaceutical composition is expected to provide the following benefits to a patient suffering cardiac injury: (1) decreased catecholamine-induced Gs protein-dependent cardiotoxicity to decrease cell death; (2) active engagement of βarrestin-dependent survival signaling to promote cell survival, thereby decreasing cell death-induced detrimental myocardial remodeling; and (3) increased cardiomyocyte contractility to actively improve cardiac function. In certain embodiments, the pharmaceutical composition can be formulated to be administered to a patient by IV injection for non-specific administration, into a lipid bilayer delivery system (i.e. exosome or immunoliposome) for targeted delivery to the heart, into a viral or non-viral delivery system for targeted delivery to the heart.

A pharmaceutical composition comprising ICL1-9 have a sequence SEQ ID No. 1, wherein said composition stimulates cardiomyocyte contractility and activating the β2AR/β-arrestin signaling pathway. In certain embodiments, the composition further simultaneously prevents cardiotoxic G protein-dependent βAR signaling. In certain embodiments the composition further promotes pro-survival signaling of cardiomyocyte cells.

Further embodiments or formulations comprising an active agent selected from the group consisting of ICL1-4, ICL1-11, ICL1-20, or ICL1-9, or combinations thereof. Such formulations include variations of each of ICL1-4, ICL1-11, ICL1-20, or ICL1-9 having 90% homology to each of these pepducins.

A single formulation may further comprise one of the ICL1-4, ICL1-11, ICL1-20, or ICL1-9 agents or combinations thereof, as well as a specific β1AR inhibitor such as bisoprolol or metoprolol. Alternatively, the β1AR inhibitor such as bisoprolol or metoprolol may be independently administered with the ICL1-4, ICL1-11, ICL1-20, or ICL1-9, or combinations thereof formulation. Furthermore, a method of treatment may comprise administering to a patient an effective amount of a composition comprising ICL1-9, ICL1-4, ICL1-11, ICL1-20 or combinations thereof, as well as a specific (31AR inhibitor for treatment of one or more of cardiovascular disease, heart disease, myocardial infarction, ischemia, reperfusion injury, AHF, CHF, and other acute and chronic diseases.

Certain embodiments are directed to the use of a composition as described herein, effective for the treatment of cardiovascular disease. Accordingly, ICL1-9 for use in the treatment of cardiovascular disease; wherein the ICL1-9 is administered to the patient in an effective dose for treatment of the cardiovascular disease.

Further embodiments comprising ICL1-4, ICL1-11, ICL1-20, or ICL1-9, or combinations thereof for use in the treatment of cardiovascular disease; wherein the ICL1-4, ICL1-11, ICL1-20, or ICL1-9, or combinations thereof, is administered to the patient in an effective dose for treatment of the cardiovascular disease.

A pharmaceutical composition comprising pepducin ICL1-9 (SEQ ID No. 1), SEQ ID No. 2, SEQ ID No. 3, SEQ ID No. 4, or combinations thereof for use as a medicament for treatment of one or more of heart disease, myocardial infarction, ischemia, reperfusion injury, AHF, CHF, and other acute and chronic diseases.

Use of pepducin ICL1-9 for the manufacture of a medicament for therapeutic treatment of heart disease, myocardial infarction, ischemia, reperfusion injury, AHF, CHF, and other acute and chronic diseases. Alternatively, ICL1-4, ICL1-11, ICL1-20, or ICL1-9, or combinations thereof may be utilized for the manufacture of a medicament for therapeutic treatment of heart disease, myocardial infarction, ischemia, reperfusion injury, AHF, CHF, and other acute and chronic diseases.

### BRIEF DESCRIPTION OF THE DRAWINGS

FIG. 1 depicts the ability of pepducins from the first intracellular loop of the β2AR to selectively promote β-arrestin binding to the β2AR (panel A) with affinities in the range of 100 nM to 1.7 μM (panel B). The most potent pepducin (ICL1-9) does not promote any cAMP production (panel C).

FIG. 2 demonstrates that ICL1-9 promotes β2AR phosphorylation (panels A and B) and internalization (panel C).

FIG. 3 demonstrates that ICL1-9 is specific towards the β2AR and has no effect on CXCR4 (panel A) or the β1AR (panels B and C).

FIG. 4 demonstrates that ICL1-9 promotes β-arrestin-biased signaling through ERK1/2 (panel A) and EGF receptor transactivation (panel B).

FIG. 5 demonstrates that ICL1-9 operates independently of the orthosteric ligand binding site (panel A) and is able to stabilize a β2AR conformation that can interact with β-arrestins (panels C and D). The ICL1-9 induced β2AR conformation can be inhibited by a potent inverse agonist (panel B).

FIG. 6 demonstrates that ICL1-9 promotes a β2AR-dependent cardiomyocyte contraction while carvedilol does not have a similar efficacy.

FIG. 7 demonstrates that ICL1-9 does not activate a calcium flux (panels A and B) or phosphorylation of phospholamban (panel C).

FIG. 8 demonstrates that ICL1-9 activation of cardiomyocyte contraction is dependent on the β2AR and β-arrestin1.

FIG. 9 demonstrates that ICL1-9 decreases infarct size following ischemia-reperfusion (FR) injury in an in vivo mouse model.

FIG. 10 demonstrates that ICL1-9 decreases cell death and improves contractile function in mice receiving ischemia-reperfusion (I/R) injury in vivo.

### DETAILED DESCRIPTION OF THE EMBODIMENTS

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 5% of the numerical value of the number with which it is being used. Therefore, about 50% means in the range of 45%-55%.

“Administering” when used in conjunction with a therapeutic means to administer a therapeutic directly to a subject, whereby the agent positively impacts the target. “Administering” a composition may be accomplished by, for example, injection, oral administration, topical administration, or by these methods in combination with other known techniques. Such combination techniques include heating, radiation, ultrasound and the use of delivery agents. When a compound is provided in combination with one or more other active agents (e.g. other anti-atherosclerotic agents such as the class of statins), “administration” and its variants are each understood to include concurrent and sequential provision of the compound or salt and other agents.

By “pharmaceutically acceptable” it is meant the carrier, diluent, adjuvant, or excipient must be compatible with the other ingredients of the formulation and not deleterious to the recipient thereof.

“Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound o the present invention and a pharmaceutically acceptable carrier.

As used herein, the term “agent,” “active agent,” “therapeutic agent,” or “therapeutic” means a compound or composition utilized to treat, combat, ameliorate, prevent or improve an unwanted condition or disease of a patient. Furthermore, the term “agent,” “active agent,” “therapeutic agent,” or “therapeutic” encompasses a combination of one or more of the compounds of the present invention.

A “therapeutically effective amount” or “effective amount” of a composition is a predetermined amount calculated to achieve the desired effect, i.e., to inhibit, block, or reverse the activation, migration, proliferation, alteration of cellular function, and to preserve the normal function of cells. The activity contemplated by the methods described herein includes both medical therapeutic and/or prophylactic treatment, as appropriate, and the compositions of the invention may be used to provide improvement in any of the conditions described. It is also contemplated that the compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes prolonging survival as compared to expected survival if not receiving treatment.

β-antagonists, also known as β-blockers, have been indicated for the treatment of pathological cardiac diseases, including congestive heart failure (CHF) and high blood pressure, for decades (1, 2). A select number of these agents, including the clinically used carvedilol, have been identified as β-arrestin biased agonists of β-adrenergic receptors based on their ability to promote β-arrestin dependent signaling over G protein activation (3, 4). It is believed that the β-arrestin activation may provide additional cardioprotection based on its ability to mediate anti-apoptotic signaling. As these are orthosteric ligands, there have been no means to decouple the activation of receptor-dependent β-arrestin signaling from the occupation of the orthosteric ligand binding pocket to study their independent contribution to its efficacy as these properties appear inherently linked.

β-blockers act to block excessive catecholamine stimulation of βARs to decrease cellular apoptotic signaling and normalize β1AR expression and inotropy. While these actions reduce cardiac remodeling and mortality outcomes, the effects are not sustained. Converse to G protein-dependent signaling, 3-arrestin-dependent signaling promotes cardiomyocyte survival. Given that β2AR expression is unaltered in CHF, a β-arrestin-biased agonist that operates though the β2AR represents a potentially useful and novel therapeutic approach.

While current β-blockers act to shield βARs from chronically elevated levels of cardiotoxic catecholamines in the treatment of heart failure, they have a significant side effect in that they inhibit cardiomyocyte contractility and thus decrease normal heart function. This is especially of concern in decompensated heart failure where PAR-mediated inotropic reserve is essential for maintenance of cardiac function. For example, such inhibition may reduce cardiac performance and result in fatigue, dizziness and weakness. By comparison, the pepducin described herein does not inhibit cardiac function and instead stimulates cardiomyocyte contractility while simultaneously preventing cardiotoxic G protein-dependent βAR signaling and promoting β-arrestin-dependent pro-survival signaling. Accordingly, by eliminating significant side effects of currently prescribed β-blockers, while also activating the β2AR/β-arrestin signaling pathway, applicant has identified a superior composition that can be utilized to treat any number of cardiac diseases.

In order to understand the relative contribution of β-arrestin-bias to the efficacy of select β-blockers, a specific β-arrestin-biased pepducin for the β2AR, ICL1-9, was used to decouple β-arrestin-biased signaling from occupation of the orthosteric ligand binding pocket. With similar efficacy to carvedilol, ICL1-9 was able to promote β2AR phosphorylation, β-arrestin recruitment, β2AR internalization, and β-arrestin-biased signaling. Interestingly, ICL1-9 was also able to induce β2AR-dependent contractility in primary adult murine cardiomyocytes while carvedilol had no efficacy. ICL1-9 promoted contractility was independent of calcium and cAMP but dependent on the β2AR and β-arrestin1.

ICL1-9 is derived from intracellular loop 1 (ICL1) of the human β2AR. Pepducin synthesis was performed by a standard Fmoc (N-(9-fluorenyl) methoxycarbonyl) solid-phase protocol with an N-terminal palmitoylation and C-terminal amidation. ICL1-9 is defined herein as SEQ ID No. 1: TAIAKFERLQTVTNYFIT.

Thus, ICL1-9 is the first reported molecule to access a pharmacological profile stimulating cardioprotective signaling and inotropic effects through the β2AR and serves as a model for next generation cardiovascular drug development. Indeed, ICL1-9 was investigated and the results provided for a characterization of a library of modulators of the β2-adrenergic receptor (β2AR) known as pepducins (5). Pepducins are lipidated peptides derived from the intracellular loops of a G protein-coupled receptor (GPCR) that can stimulate or inhibit downstream signaling processes of their cognate receptor (6). From a two-dimensional screen, the β2AR pepducin library displayed a wide-range of properties, spanning those that had complete Gs-bias to some that were β-arrestin-biased (5).

Therefore, as described herein, ICL1-9, a β-arrestin-biased pepducin derived from the PAR, is used to dissect the relative contribution of β-arrestin-bias in the bipartite mechanism of clinically relevant β-blockers. In certain preferred embodiments, described herein, ICL1-9 is able to effectively promote the activities expected of a β-arrestin-biased agonist including GRK-mediated receptor phosphorylation, β-arrestin recruitment, receptor desensitization, receptor internalization, ERK activation and EGF receptor transactivation comparable to the reported efficacy of carvedilol. As these actions are independent of the orthosteric ligand binding site, ICL1-9 is a unique tool in which the contribution of β-arrestin processes and signaling of a β-arrestin-biased β-blocker can be assessed in isolation.

As described in the examples below, a comparative functional study between carvedilol and ICL1-9 was performed to assess their relative efficacy in regulating primary murine cardiomyocyte contractility. Surprisingly, ICL1-9 was able to induce cardiomyocyte contraction while carvedilol did not. Taken together, a β2AR-specific β-arrestin biased pepducin is characterized that promotes known cardioprotective signaling pathways paired with induction of cardiomyocyte contractility. This pharmacological profile is not only the first to be reported through the β2AR but may provide a therapeutically superior alternative to currently prescribed β-blockers, which protect the heart against catecholamine toxicity, but do not actively engage pro-survival or pro-contractile pathways at therapeutically relevant doses to enhance cardiac function.

cAMP Measurement.

HEK293 cells stably overexpressing a FLAG-β2AR were cultured to confluency in 24 well plates at 37° C. in Dulbecco's Modified Eagle Medium (DMEM, Cellgro) supplemented with 10% fetal bovine serum (FBS) and 50 μg/ml G418 sulfate (Cellgro). Cells were stimulated with 100 pM to 100 μM isoproterenol or ICL1-9 for 10 min at 37° C. in the presence of 0.5 mM 3-isobutyl-1-methylxanthine (IBMX). Stimulation was ended by the removal of media on ice and cells were lysed by adding 80 μl 0.1 M HCl followed by 20 min incubation at room temperature on an orbital shaker. Lysates were cleared by centrifugation at 1,000×g for 15 min. cAMP levels were measured using the Cayman Chemical Cyclic AMP EIA kit according to the manufacturer's instructions.

β-Arresting Recruitment Using Bioluminescence Resonance Energy Transfer.

β-arrestin2 recruitment to the β2AR was measured as previously described (5). In brief, HEK293 cells co-expressing β-arrestin2-GFP10 (energy acceptor) and β2AR-RLucII (energy donor) were stimulated with 100 pM to 100 μM isoproterenol or ICL1-9 in the presence of 2.5 μM Coelenterazine 400a. BRET was monitored over the course of 24 min using a Tecan Infinite F500 microplate reader. BRET ratios were calculated as the light intensity emitted by GFP10 at 510 nm divided by the light emitted by the donor RLucII at 400 nm. The background of unstimulated trials was calculated from the BRET measured from the stimulated trials to report ABRET.

β-arrestin2 recruitment to CXCR4 was measured similarly in HEK293 cells stably overexpressing a FLAG-β2AR and transiently transfected with β-arrestin2-GFP10 and CXCR4-RLucII (15). BRET was monitored post-stimulation using 50 nM SDF-1α, 1 μM isoproterenol or 10 μM ICL1-9.

Detection of β₂AR Phosphorylation Using Phosphospecific Antibodies.

HEK293 cells stably overexpressing FLAG-β₂AR were grown in 10 cm dishes at 37° C. in DMEM supplemented with 10% FBS and 50 μg/ml G418 sulfate (Cellgro). Cells were stimulated with 1 μM isoproterenol, 10 μM carvedilol or 10 μM ICL1-9 for 0-60 min at 37° C. and the cells were washed, lysed and then analyzed for β2AR phosphorylation at Ser³⁵⁵ and Ser³⁵⁶ as previously described (5). Briefly, cell lysates were immunoprecipitated using mouse monoclonal M2 anti-FLAG (Sigma-Aldrich) and Protein G agarose PLUS beads (Santa Cruz Biotechnologies). The beads were incubated overnight at 4° C., pelleted, washed and then suspended in Laemeli buffer. Immunoprecipitated proteins were separated by SDS-PAGE and receptor phosphorylation was analyzed by western blotting using a phosphospecific antibody (1:500) against β₂AR phospho-Ser^{355/356} (Santa Cruz Biotechnologies). Chemiluminescence was measured using Pico chemiluminescent substrate (Thermo Scientific).

β-Adrenergic Receptor Internalization by Cell Surface ELISA.

Receptor internalization was measured by cell surface enzyme-linked immunosorbent assay as previously described (5, 45).

β1AR/β-Arrestin2 Interaction Measurements by Fluorescent Resonance Energy Transfer (FRET).

Human osteosarcoma (U2S) cells were seeded on fibronectin (10 μg/ml)-coated glass coverslips in 35 mm dishes in MEM containing 10% FBS and 1% penicillin/streptomycin/amphotericin B and infected with adenoviral constructs for Flag-β1AR-mCFP (MOI of 60) and Ad-βarrestin2-mYFP (MOI of 200). 24 h following infection, cells were rinsed and media replaced with imaging buffer (HBSS supplemented with 0.2% BSA and 20 mM HEPES) 10 min prior to imaging using a Leica DMI4000B inverted microscope with a Leica DFC365 FX 1.4-megapixel monochrome digital camera. CFP (433/475 nm), YFP (514/527 nm) and FRET (433/527 nm) excitation and emission wavelengths were measured every 3.5 sec. After 30 sec of baseline reads the cells were stimulated with isoproterenol (1 μM) or ICL1-9 (10 μM) and whole field-of-view measurements at 20× magnification were used to assess changes in FRET. Quantification of the changes in FRET (corrected FRET=FRET−(CFP*CFP bleed-through [36%])−(YFP*YFP bleed-through [13%]) were expressed as a % of total CFP emission (% FRET=cFRET/[cFRET+CFP]).

Detection of ERK Phosphorylation and EGFR Transactivation.

HEK293 cells stably overexpressing FLAG-β2AR were grown to ˜90% confluence in 6 well plates and serum starved for 16 h. Cells were stimulated with 10 μM carvedilol or 10 μM ICL1-9 over a 1 h time-course at 37° C. in 0.05% DMSO in non-pepducin trials. On ice, assay media was removed and 100 μl of lysis buffer was added. Cell lysates were scraped and briefly sonicated. 20 μl of 6× Laemmli buffer was added and the lysate was boiled for 10 min. ERK phosphorylation was detected by western blotting using a polyclonal primary antibody against phospho-ERK1/2 (1:500 in TBST with 5% BSA, Cell Signaling Technologies) and total ERK2 levels were detected using a monoclonal anti-ERK2 antibody (1:1000 in TBST with 5% BSA, Santa Cruz Biotechnologies). ERK phosphorylation levels (normalized to ERK2) were quantitated by detection of anti-mouse IRDye 800 and anti-rabbit IRDye 680 antibodies using a LiCOR Odyssey system.

[125I]-Iodocyanopindolol Binding.

HEK293 cells stably expressing a FLAG-β2AR were isolated and washed 3 times with assay buffer (HBSS with calcium and magnesium, 0.1% BSA, pH 7.4), diluted to 25,000 cells/ml and incubated with 1 nM [125I]-iodocyanopindolol in the presence or absence of pepducin or carvedilol for 2 h at 25° C. Incubations were terminated by rapid filtration on GF/B filters. Filters were washed 4 times with 5 ml of cold assay buffer and [125I]-iodocyanopindolol binding was quantitated by gamma emission counting.

β-Arrestin Coupling Assessed by Monobromobimane Fluorescence.

Full-length PN1-β2AR was purified from Sf9 insect cells and labeled with monobromobimane as previously described (5, 46). Monobromobimane-labeled β2AR was reconstituted in 2% DOPC/CHAPSO (3:1) with 1.13 mM CHS lipid bicelles by incubating for 30 min on ice. Lipid bicelles containing 50 nM mBB-β2AR were incubated for 15 min at 25° C. in 20 mM HEPES, pH 7.5, 100 mM NaCl with 10 μM carvedilol or 10 μM ICL1-9. In experiments using β-arrestin1, 200 nM WT β-arrestin1 or 200 nM AAF-mutant β-arrestin1 was incubated for 10 min at 25° C. alone or post-agonist addition depending on experimental set-up. mBB-β2AR fluorescence was measured by excitation at 370 nm and recording emission from 430-490 nm at 1 nm increments with 1 nm s-1 integration on a Perkin Elmer LS55 fluorescence spectrophotometer set at a 5 nm emission bandwidth pass. Background fluorescence contributed by the assay buffer and ligand were subtracted from the experimental spectra.

Isolation of Adult Murine Cardiac Myocytes, Contractility and Ca²⁺ Measurements.

Adult murine cardiac myocytes were isolated from the septum and LV free wall of 8-12 week old mice as previously described (47). Briefly, mice were heparinized (1,500 U/kg ip) and anesthetized (pentobarbital sodium, 50 mg/kg ip). Excised hearts were mounted on a steel cannula and retrograde perfused (100 cm H2O, 37° C.) with Ca2+-free bicarbonate buffer followed by enzymatic digestion (collagenases B and D, protease XIV). Isolated myocytes were plated on laminin-coated glass coverslips, and the Ca2+ concentration of the buffer was incrementally increased (0.05, 0.125, 0.25, 0.5 mM) with 10 min of exposure at each concentration. The final Ca2+ buffer was then aspirated and replaced with MEM (Sigma-Aldrich) containing 1.2 mM Ca2+, 2.5% FBS, and 1% penicillin/streptomycin. The pH was adjusted to 7.0 in 4% CO2 by the addition of NaHCO₃ (0.57 g/l). After 1 h (4% CO2, 37° C.), media was replaced with FBS-free MEM containing 0.1 mg/ml BSA and antibiotics. Myocytes adherent to coverslips were bathed in 0.7 ml of air- and temperature-equilibrated (37° C.) HEPES-buffered (20 mM, pH 7.4) medium 199 containing 1.8 mM [Ca2+] and used within 2 to 8 h of isolation. For Ca²⁺ transient measurements, cardiomyocytes were exposed to 0.67 Fura 2-AM for 15 min at 37° C. Measurements of myocyte contraction at a pacing frequency of 2 Hz were performed in the presence of vehicle (0.1% DMSO), isoproterenol (0.5 μM), ICL1-9 (10 μM), control pepducin (10 μM) or carvedilol (10 μM).

Detection of β-Arrestin Expression and Phospholamban Phosphorylation.

Isolated cardiomyocytes (prepared as described above) were stimulated with 0.1% DMSO, 0.1 μM isoproterenol or 10 μM ICL1-9 for 5 min. On ice, assay media was removed and 100 μl of lysis buffer was added, cells were scraped and or mutated at 4° C. for 30 min. 20 μl of 6× Laemeli buffer was added and the lysate was boiled for 10 min. Left ventricular samples were homogenized in lysis buffer containing 20 mM Tris-HCl, pH 7.4, 137 mM NaCl, 10% glycerol, 1 mM EDTA, 1% NP-40, 10 mM NaF (Fisher Scientific, Pittsburgh, Pa.), 1×HALT protease inhibitor cocktail (Thermo Scientific, Rockford, Ill.) and phosphatase inhibitor cocktail set IV (Calbiochem, USA). Lysates were run on 8% SDS-PAGE gels and transferred to Immobilon-PSQ polyvinylidene fluoride 0.2 mm pore size membranes (Millipore, Billerica, Mass.). PLB phosphorylation (cardiomyocyte lysates) was detected using anti-Phospho-Ser¹⁶ PLB rabbit pAb (1:5000, Badrilla) and normalized to total PLB as detected with anti-PLB mouse mAb (1:1000, Badrilla). β-arrestin1 and 2 expression levels (left ventricular lysates) were detected using anti-β-arrestin1/2 rabbit mAb (1:1000, Cell Signaling Technology) and normalized to GAPDH levels as detected with anti-GAPDH rabbit mAb (1:1000, Cell Signaling Technology). Membranes were subsequently incubated with appropriate anti-rabbit or anti-mouse IRDye (680 or 800)-labeled antibodies and detected using the LiCOR Biosciences Odyssey system.

Analysis of Infarct Size and Cell Death Following Ischemia-Reperfusion (I/R) Injury in Mice.

ICL1-9 or scrambled pepducin (1 ng/μL per 10 μL injection) were injected into the left ventricular wall of C57Bl/6 mice at three sites just prior to sham surgery or left descending coronary artery ligation for 60 min. After 24 hr of reperfusion, the hearts were perfused with Evans Blue dye and the mice were sacrificed, hearts frozen, stained with TTC, photographed and risk and infarct areas calculated. Treated mice were also analyzed after 24 hr reperfusion for cell death staining (TUNEL, red), nuclear staining (DAPI, blue) and cardiomyocyte staining (α-sarcomeric actin, green) of cardiac slices.

Analysis of Cardiac Function Following Ischemia-Reperfusion (I/R) Injury in Mice.

Cardiac function was assessed via transthoracic two-dimensional echocardiography performed at baseline and at 1, 3 and 7 days post MI using a 12-mHz probe on mice anesthetized with isoflurane (1.5%). M-mode echocardiography was performed in the parasternal short-axis view to assess several cardiac parameters including left ventricular (LV) fractional shortening, calculated using the equation ((LVID;d-LVID;s)/LVID;s)*100%.

Results

ICL1-9 is a Potent β-Arrestin-Biased Pepducin

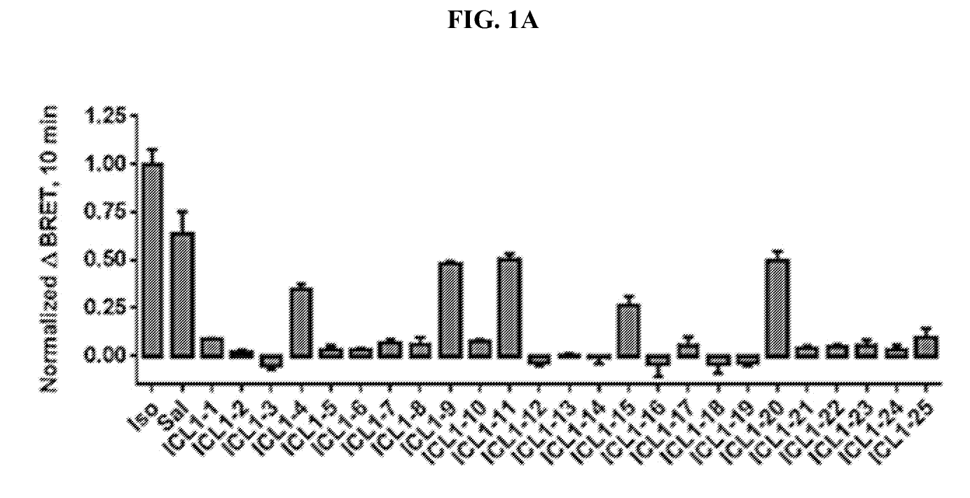

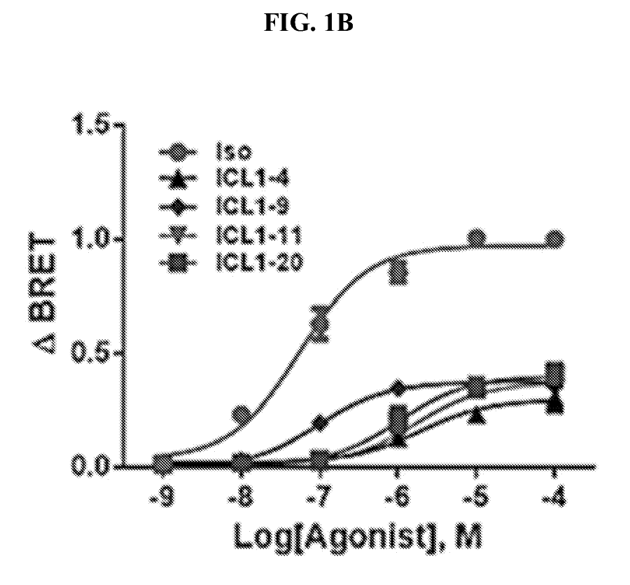

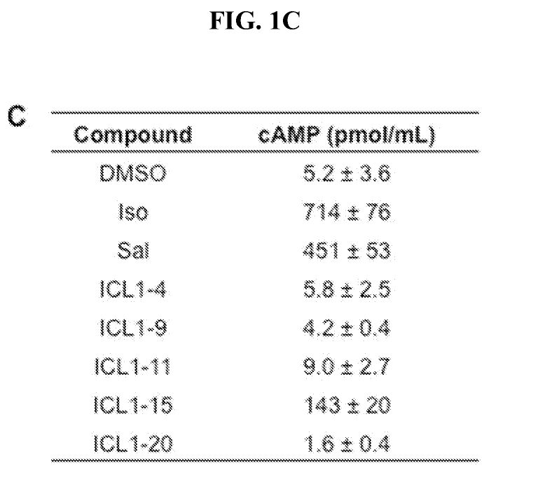

In initial characterizations of the β2AR-derived pepducin library, it appeared that putative ‘β-arrestin-biased’ pepducins were derived from ICL1. To assess pepducin-promoted β-arrestin recruitment, bioluminescence resonance energy transfer (BRET) was monitored in HEK293 cells co-transfected with a β2AR-Renilla Reniformus luciferase II fusion (β2AR-RLucII) and GFP10-tagged β-arrestin2. At 10 min, pepducins ICL1-4, ICL1-9, ICL1-11, ICL1-15 and ICL1-20 were able to promote significant β-arrestin recruitment with efficacies ranging between 23%-48% of the response to isoproterenol (a non-selective β-agonist) (FIG. 1A). The data are represented by the mean±SD from three independent experiments. As ICL1-15 has been previously demonstrated to promote modest increases in cAMP production (5), and prolonged PAR-mediated cAMP signaling in the heart is not a desirable property since it is associated with increased mortality levels in human HF patients (48), it was not studied further. FIG. 1B depicts that ICL1-9 is a high-potency β-arrestin-biased pepducin with an EC₅₀ of 95±14 nM. ICL1-4 (1.9±0.5 μM), ICL1-11 (1.7±0.5 μM) and ICL1-20 (1.1±0.3 μM) demonstrated comparable efficacy to ICL1-9 but operated with lower potency. The β-arrestin-bias of these pepducins was verified by analysis of cAMP production in HEK293 cells stably overexpressing a FLAG-β₂AR. ICL1-4, -9 and -20 did not promote any cAMP production compared to vehicle control while ICL1-11 gave a ˜2-fold increase (FIG. 1C). ICL1-4 corresponds to SEQ ID. No. 2; ICL1-9 corresponds to SEQ ID. No. 1; ICL1-11 corresponds to SEQ ID No. 3, and ICL1-20 corresponds to SEQ ID No. 4. This compares with isoproterenol and salbutamol which gave 137-fold and 87-fold increases in cAMP, respectively. Thus, ICL1-9 is a potent β-arrestin-biased activator of the β₂AR and was used for additional characterization and mechanistic studies.

ICL1-9 Exhibits the Functional Properties of β-Arrestin Bias.

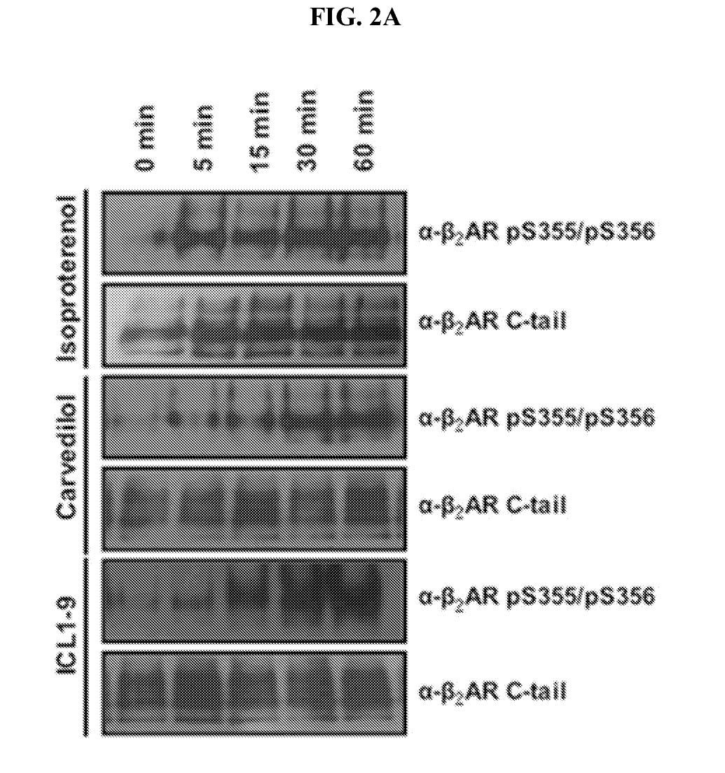

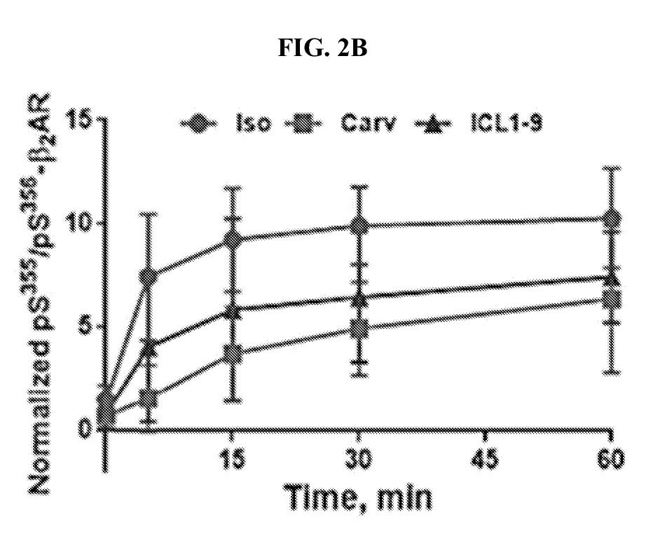

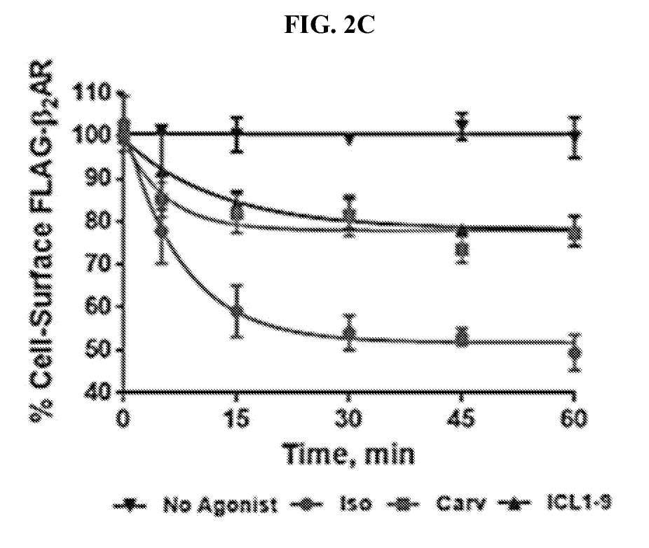

β-arrestin recruitment is dependent on GRK-mediated phosphorylation of the C-terminal tail of the γ2AR (7). Agonist-promoted GRK-mediated β2AR phosphorylation was assessed using a phosphospecific antibody detecting phosphorylation of ³⁵⁵Ser/³⁵⁶Ser in HEK293 cells stably expressing FLAG-β2AR (8-10). Isoproterenol rapidly and robustly promoted phosphorylation at this site while both carvedilol (a non-selective β-arrestin-biased agonist) and ICL1-9 stimulated similar receptor phosphorylation albeit with slower kinetics and extent of phosphorylation (FIGS. 2A and B). It appears that the pepducin may stabilize a PAR conformation that is a favorable substrate for GRKs and subsequent β-arrestin recruitment.

FIG. 2 depicts that ICL1-9 promotes β2AR phosphorylation, internalization and desensitization. FIGS. 2A and B depicts receptor phosphorylation monitored over a time-course in the presence of 1 μM isoproterenol, 10 μM carvedilol or 10 μM ICL1-9 in HEK293 cells stably overexpressing a FLAG-β2AR. In-cell phosphorylation was detected using a phosphospecific antibody for pSer³⁵⁵/pSer³⁵⁶ post-receptor immunoprecipitation. With slower kinetics than a β-agonist (isoproterenol), ICL1-9 and carvedilol promoted robust receptor phosphorylation. The data are representative of three independent experiments.

FIG. 2C depicts that both carvedilol and ICL1-9 were able to stimulate comparable levels of FLAG-β2AR internalization as monitored by a cell-surface ELISA assay, albeit less than that induced by isoproterenol. The data are represented by the mean±SD from three independent experiments.

β-arrestins are critical regulators of agonist-promoted receptor internalization for many GPCRs including the β2AR (11). Receptor internalization of the β2AR was studied by cell surface ELISA post-stimulation using isoproterenol, carvedilol, and ICL1-9. As expected, isoproterenol, carvedilol and ICL1-9 were able to promote β2AR internalization with variable kinetics and efficacy (FIG. 2C).

ICL1-9 Demonstrates Selectivity for the β2AR.

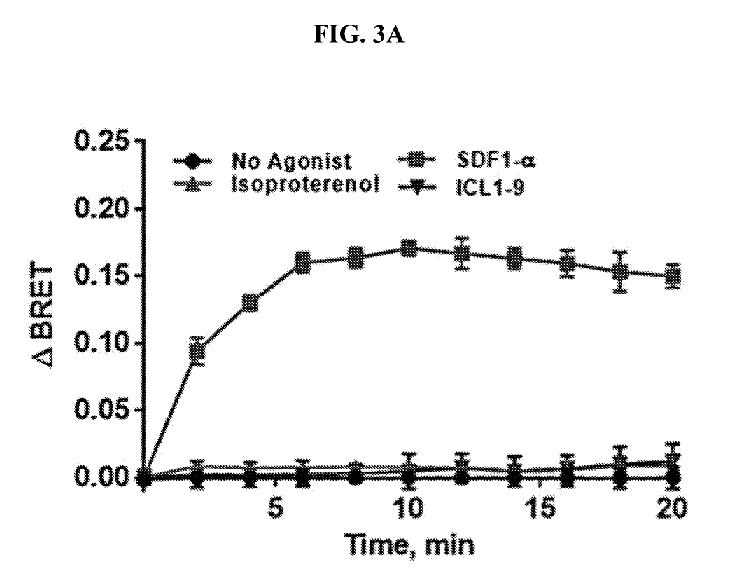

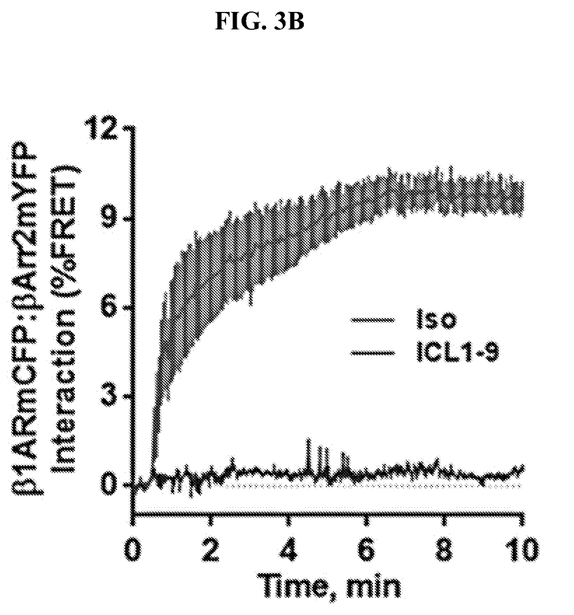

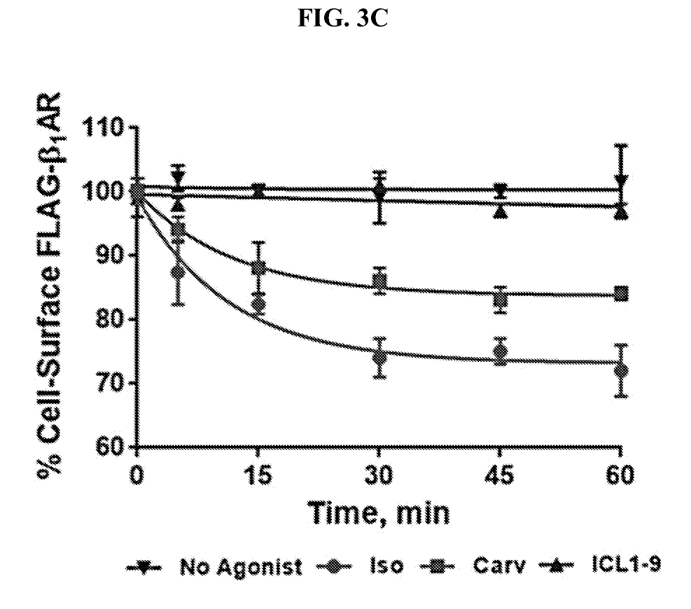

It was believed that pepducins demonstrate receptor specificity for the cognate receptor in which it was derived (6). However, there is growing evidence that some pepducins can operate through multiple GPCRs as some pepducin sequences can be found in multiple receptor subtypes (5, 12-14). It is also plausible that ICL1-9 is operating independently of a particular receptor and directly recruiting β-arrestins to the cell membrane. This mode of operation may crowd the membrane with the BRET acceptor and create a ‘false-positive’ profile for specific BRET interactions that could be concluded at any receptor of interest. FIG. 3 provides evidence that ICL1-9 demonstrates specificity towards the PAR.

FIG. 3A depicts β-arrestin2 recruitment monitored over a time-course post-agonist stimulation with 50 nM SDF-1α, 1 μM isoproterenol or 10 μM ICL1-9 by BRET² in HEK293 cells stably overexpressing a FLAG-β2AR and transiently transfected with CXCR4-RLucII and GFP10-β-arrestin2. SDF-la was able to effectively promote β-arrestin2 recruitment to CXCR4 while isoproterenol and ICL1-9 had no effect. The data are represented by the mean±SD from three independent experiments. These results suggest that ICL1-9 promotes a β₂AR-dependent interaction with β-arrestins and does not operate via direct recruitment of β-arrestin to the cell membrane.

Due to sequence similarity of the β1AR and β2AR (71% identity and 76% similarity in ICL1; 54% identity and 61% similarity overall), it is plausible that ICL1-9 can also signal though the β1AR as ICL3-9 demonstrated in our previous report (5). In order to assess the specificity of ICL1-9 between the two-subtypes, β-arrestin recruitment to the β1AR was monitored by FRET. Agonist-promoted β-arrestin recruitment was observed when cells were treated with isoproterenol while no change in FRET activity was observed in response to ICL1-9 (FIG. 3B). Further corroborating β2AR specificity, isoproterenol and carvedilol were able to induce FLAG-β1AR internalization as monitored by a cell-surface ELISA assay while ICL1-9 did not induce significant receptor internalization over a 1 h time-course (FIG. 3C). Thus, ICL1-9 appears to be specific for the β2AR and shows no activity towards the β1AR.

Accordingly, as β1AR is implicated in mediating cell death in congestive heart failure as well as myocardial infarction and/or ischemia/reperfusion injury, in methods of treatment, as described herein, it is advantageous in certain embodiments to co-administer a β1AR inhibitor such as bisoprolol or metoprolol with the ICL1-9 pepducin.

ICL1-9 Promotes β-Arrestin Signaling.

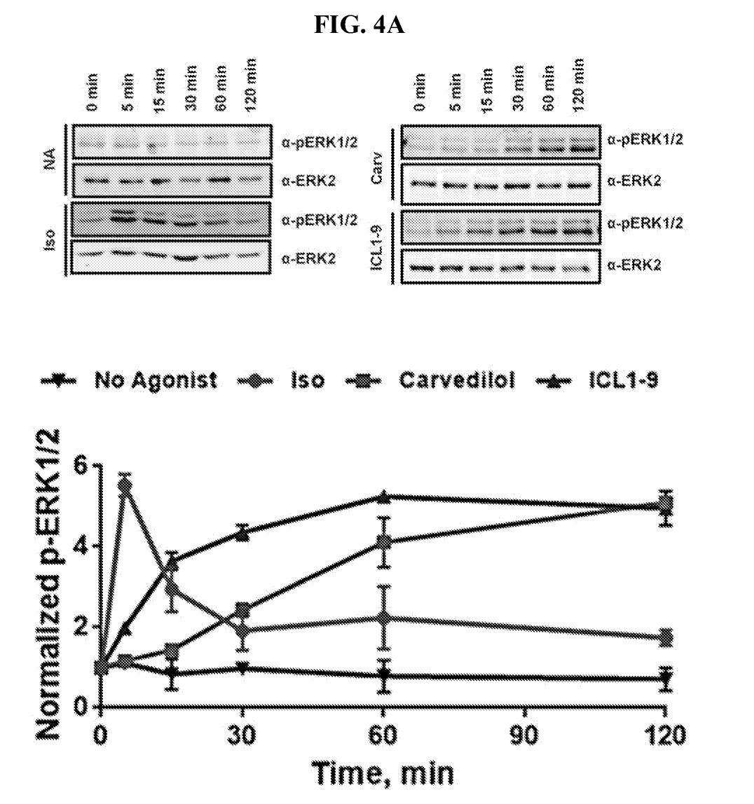

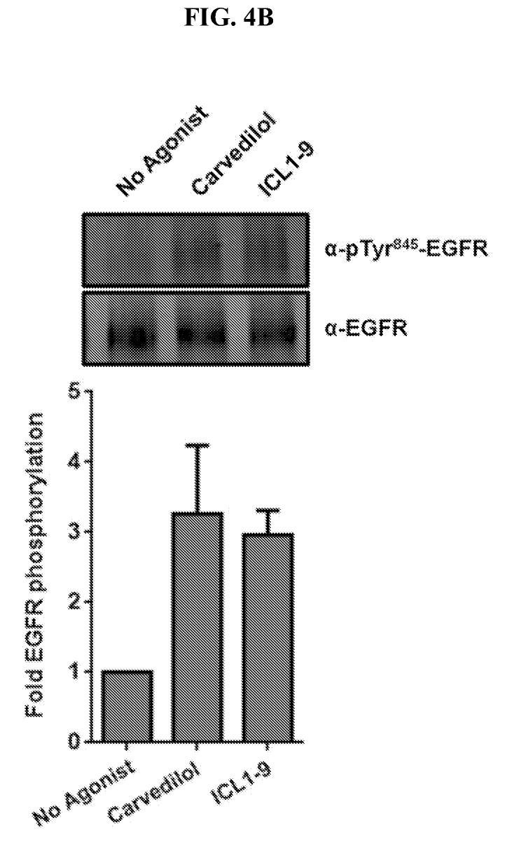

GPCRs are now appreciated to signal through a number of intracellular transducers beyond heterotrimeric G proteins including β-arrestins (7), which can act as a scaffold for multiple protein kinase cascades such as MAP kinases (16-18). Previous studies have demonstrated that isoproterenol and carvedilol can promote β-arrestin-dependent ERK1/2 phosphorylation (3). Over a 2 h time-course, ICL1-9 was able to induce ERK1/2 phosphorylation with a response profile that demonstrated faster kinetics despite similar efficacy when compared to carvedilol. Isoproterenol exhibited the fastest kinetics to maximal efficacy but lacked the magnitude of the late-phase signal observed with carvedilol and ICL1-9 stimulation (FIG. 4A). ERK1/2 phosphorylation in response to ICL1-9 was completely dependent on the expression of β-arrestins (data not shown).

One mechanism by which β-arrestin-biased β-blockers promote ERK1/2 phosphorylation involves βAR crosstalk with the epidermal growth factor receptor (EGFR) (18, 19). For example, carvedilol has been shown to promote PAR-mediated EGFR transactivation in a β-arrestin-dependent manner (20). In HEK293 cells stably overexpressing FLAG-β2AR, ICL1-9 promoted EGFR transactivation, as monitored by receptor phosphorylation at EGFR Tyr⁸⁴⁵, comparable to what is observed with carvedilol (FIG. 4B). These studies indicate that ICL1-9 and carvedilol mediate similar intracellular signaling responses although with slightly different kinetics and efficacy.

Mechanism of ICL1-9 Action.

ICL1-9 Decouples β-Arrestin-Bias Activity from the Orthosteric Ligand Binding Pocket.

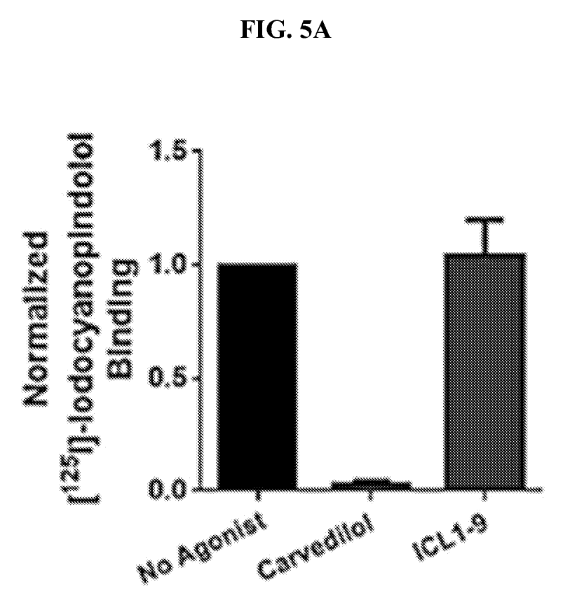

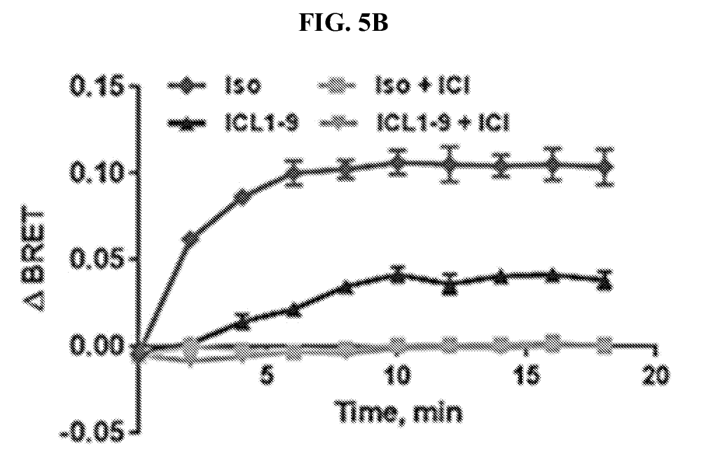

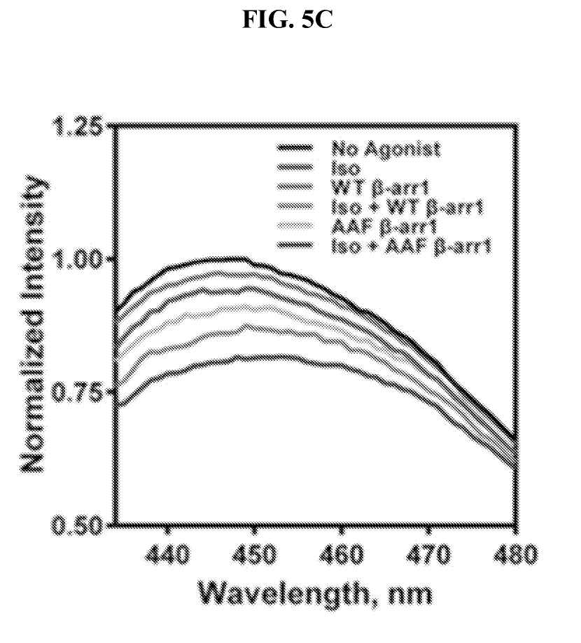

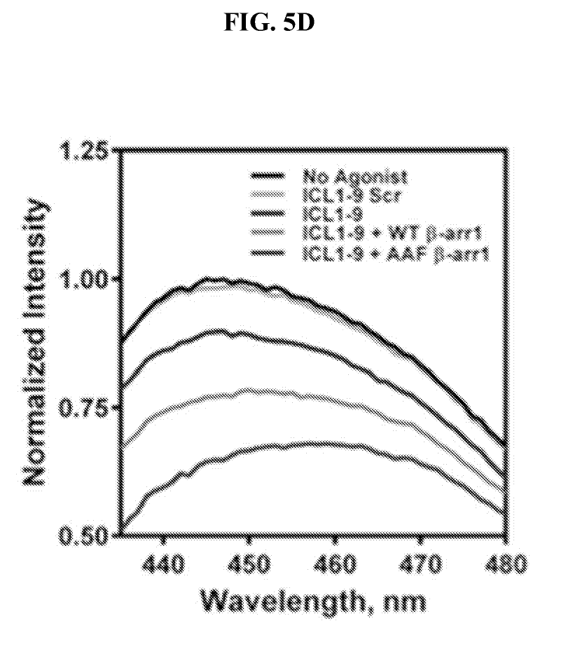

ICL1-9 can selectively promote GRK-mediated β2AR phosphorylation, β-arrestin recruitment, receptor internalization and β-arrestin-dependent signaling comparable to carvedilol. Currently, there is no method to decouple the ability of β-blockers to occupy the orthosteric ligand binding site with the ability to promote β-arrestin recruitment. To determine whether ICL1-9 acts to alter orthosteric ligand binding, competitive radioligand binding assays were performed. As expected, carvedilol effectively inhibited access to the orthosteric binding site while ICL1-9 did not affect [¹²⁵I]-iodocyanopindolol binding to β2ARs (FIG. 5A). Accordingly, ICL1-9 does not compete for the orthosteric binding site and thus would not compete for the ability of endogenous ligands such as epinephrine and norepinephrine to bind to the β2AR. Since ICL1-9 acts at β2ARs independently of the orthosteric binding site to induce β-arrestin-dependent signaling with comparable efficacy to carvedilol, it may serve as an ideal tool to understand the relative impact of β-arrestin-dependent β2AR signaling.

ICL1-9 is Sensitive to the Inverse Agonist ICI-118,551.

The inverse-agonist ICI-118,551 is proposed to operate by restricting conformational dynamics of the β2AR and stabilize an inactive receptor conformation (21-23). Thus, if ICL1-9 requires a conformational change in β2AR for activity, it may demonstrate sensitivity to ICI-118,551. Indeed, the ability of ICL1-9 to promote β-arrestin coupling to the β2AR (as monitored by BRET) was significantly inhibited by pretreatment with ICI-118,551 (FIG. 5B). A similar relationship was observed when cells were pretreated with ICI-118,551 and stimulated with isoproterenol; although, this activity can be best explained by orthosteric binding site competition. As ICL1-9 operates independently from the orthosteric ligand binding pocket, its sensitivity to ICI-118,551 likely stems from a conformational competition between an ICL1-9-promoted β-arrestin-biased conformation and an ICI-118,551-promoted inactive conformation of the β2AR.

ICL1-9 Promotes a β2AR Conformation that Couples to β-Arrestins.

Upon GRK-mediated phosphorylation, many GPCRs, including the β2AR, bind β-arrestins with high affinity (11). Partial visualization of the GPCR-arrestin interface has been recently achieved by co-crystallization studies of an arrestin-1 finger loop peptide and rhodopsin (24), and by electron microscopy and deuterium exchange analysis of a β-arrestin1 complex with a β2AR-vasopressin 2 receptor C-terminal tail fusion (25). Each study reported a number of common structural features including the stabilization of an outward movement of the receptor transmembrane 6 (TM6) by the arrestin finger loop (24). This conformational stabilization is similar to that induced by Gs interaction with the β2AR (26). Thus, it may be possible to detect β-arrestin/β2AR interaction by methods similar to those used in assessing G protein coupling.

TM6 movement associated with receptor activation and G protein interaction has been previously monitored using purified β2AR modified with a monobromobimane at Cys²⁶⁵ (mbb-β2AR) (27, 28). The environmentally-sensitive monobromobimane demonstrates a decrease in peak fluorescence and a red shift upon TM6 movement when Cys²⁶⁵ moves from a local hydrophobic environment to a position that is solvent exposed (27). Both isoproterenol and ICL1-9 were able to promote mbb-β2AR conformational changes that stabilized TM6 movement (indicated by loss of peak fluorescence and increase in λₘₐₓ; FIGS. 5C & D). Additionally, β-arrestin-promoted conformational changes were detected in TM6 as incubation with wild-type (WT) β-arrestin or β-arrestin1-AAF (a partially pre-activated mutant that promotes independence from prerequisite receptor phosphorylation (29)) modulated monobromobimane fluorescence (AAF >WT; FIG. 5C). Pretreatment with isoproterenol further stabilized TM6 movement in the presence of WT or mutant β-arrestin (FIG. 5C). Co-incubation with ICL1-9 and β-arrestins (WT and AAF) lead to striking changes in mbb-β2AR TM6 movement (FIG. 5D). While ICL1-9 stabilized β2AR/β-arrestin1 complexes exhibited greater relative changes in the Stokes shift and λₘₐₓ as compared to isoproterenol-treated complexes (FIGS. 5C & D), isoproterenol was used at a sub-saturating concentration in these studies.

ICL1-9 Promotes β2AR-Dependent Cardiomyocyte Contractility.

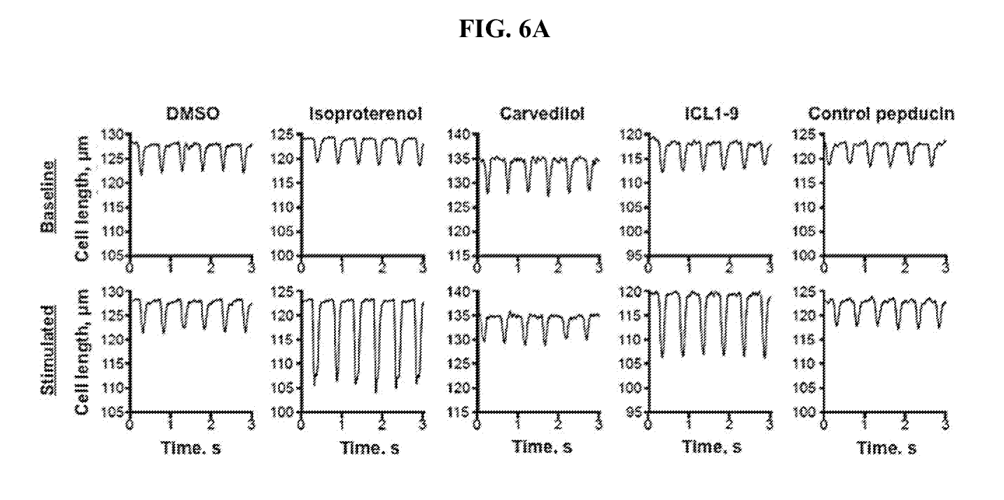

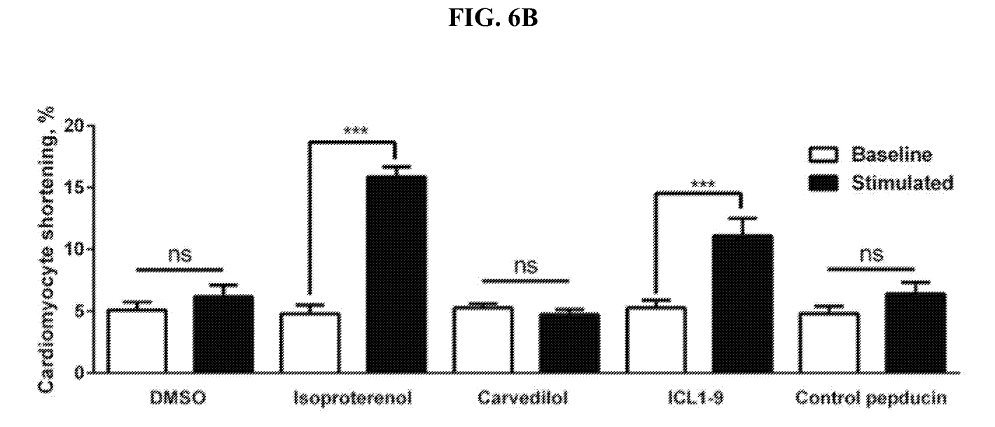

β-blockers are commonly prescribed pharmaceuticals used in the treatment of CHF (1, 2). It is believed that β-blockers act to inhibit pathogenic βAR signaling pathways, including those mediating cell death (30-32). As G protein-dependent signaling has been attributed to cardiomyocyte death, the use of a β-arrestin-biased agonist could be an advantageous therapeutic approach (33). Beyond its inability to inactivate G protein signaling, evidence has suggested that β-arrestin-biased signaling promotes cardiomyocyte survival signaling along with induction of cardiomyocyte contractility (3, 34). Indeed, a β-arrestin-biased agonist for the angiotensin II Type 1A receptor (AT1R), TRV027, has demonstrated the ability to promote β-arrestin-dependent cardiac contraction in vivo and is currently in clinical trials for the treatment of heart failure (34-37). As a comparable pharmacological profile through the β₂AR has yet to be reported, the ability to promote contraction was assessed using primary wild-type adult murine cardiomyocytes. Surprisingly, ICL1-9 was able to induce robust cardiomyocyte contraction (˜53% of isoproterenol-promoted) despite its inability to stimulate Gₛ protein activation (FIGS. 6A & B). Interestingly, when WT cardiomyocytes were treated with carvedilol, a partial restriction of basal contraction was observed rather than induction of contraction (FIGS. 6A & B).

FIG. 6A depicts wild-type adult murine cardiomyocytes that were isolated and assessed for basal and agonist-promoted contractility using a digital video camera coupled microscope in the presence or absence of 0.1% DMSO, 0.5 μM isoproterenol, 10 μM carvedilol and 10 μM ICL1-9 or control pepducin. Representative cell length (in μm) tracings at 2 Hz in the basal or stimulated state for each test condition are reported. FIG. 6B depicts that ICL1-9 was able to promote significant contraction in WT adult murine cardiomyocytes (˜53% of isoproterenol-promoted contractility) while carvedilol did not stimulate a similar effect. The data are represented by the mean±SEM from 3-8 independent experiments. ns—not significant, ***—p<0.001 using a one-way ANOVA with Newman-Keuls Multiple Comparison Test.

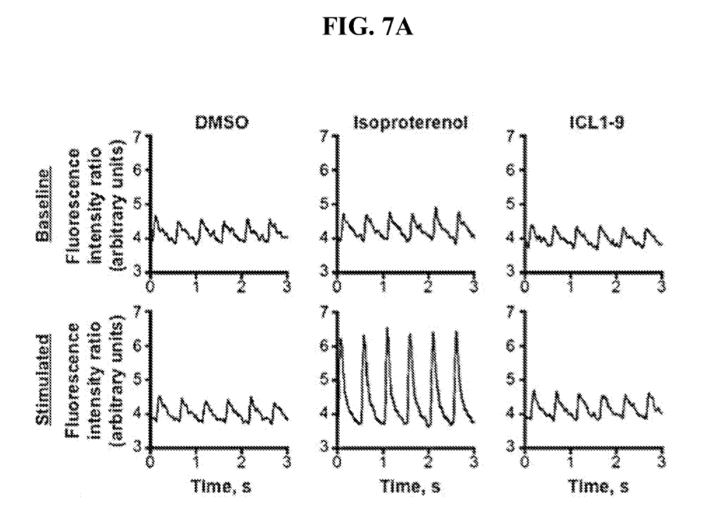

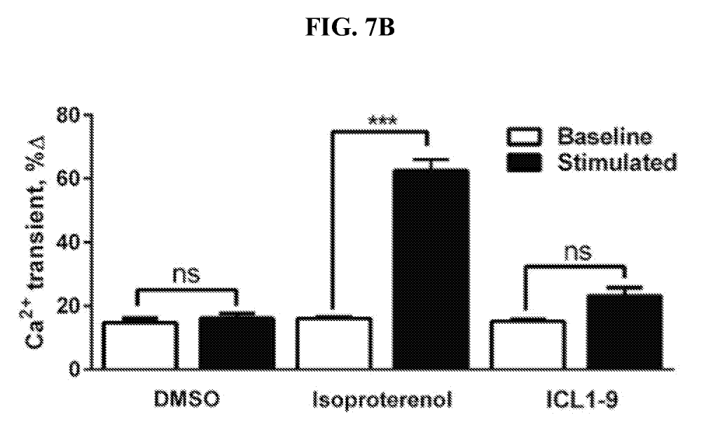

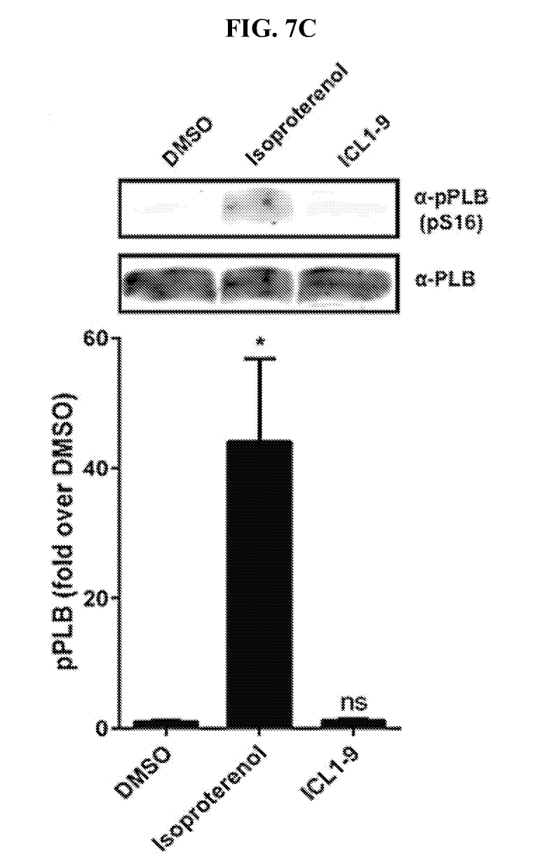

Conventional cardiomyocyte contraction mechanisms depend on calcium mobilization and subsequent activation of the cardiomyocyte sarcomere. Unlike isoproterenol, ICL1-9 did not promote significant calcium mobilization in primary murine cardiomyocytes (FIGS. 7A & B). The activation of protein kinase A (PKA) signaling also plays a prominent role in conventional cardiomyocyte inotropic mechanisms. PKA activity is dependent on the upstream generation of cAMP and phosphorylates a number of effectors involved in cardiomyocyte inotropy and lusitropy. In response to β-agonists, PKA contributes to cardiomyocyte relaxation mechanisms through the phosphorylation of phospholamban (PLB), which relieves its inhibition of the sarcoplasmic reticulum Ca²⁺-ATPase (SERCA) pump and leads to rapid calcium re-uptake into the sarcoplasmic reticulum. As ICL1-9 is proposed to operate though a β-arrestin-mediated mechanism of cardiomyocyte contraction, ICL1-9 was unable to induce significant PLB phosphorylation while isoproterenol, a conventional β-agonist, stimulated robust PLB phosphorylation (FIG. 7C).

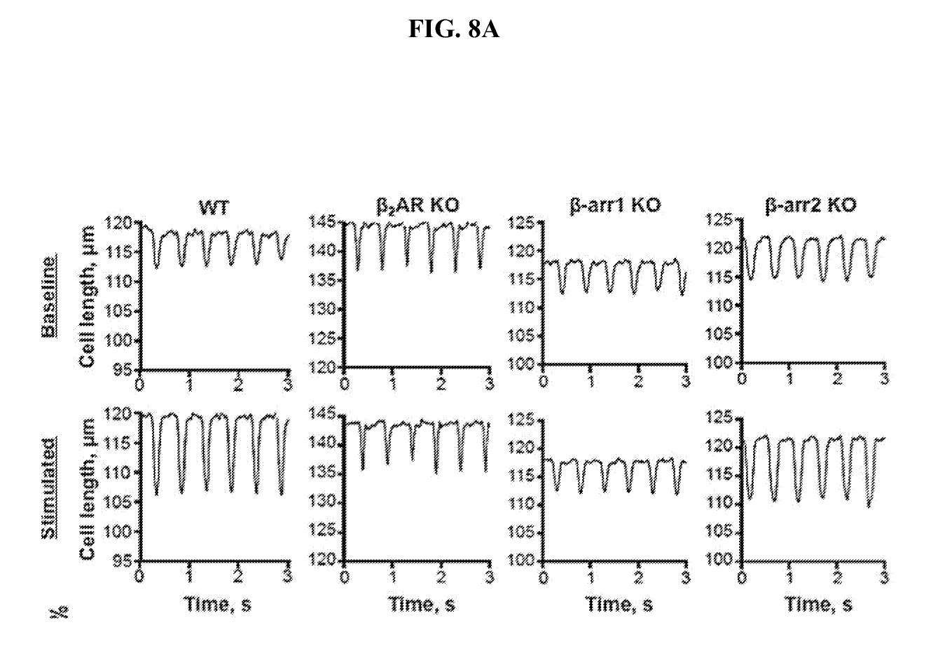

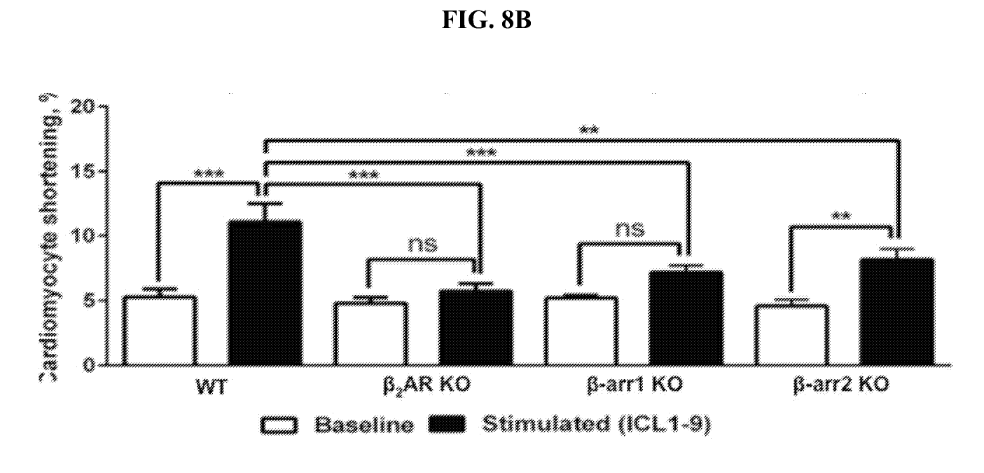

Although ICL1-9 does not promote cardiomyocyte contraction through conventional mechanisms, ICL1-9 activity is strikingly dependent on the β₂AR and operates through β-arrestin recruitment as cardiomyocytes derived from β₂AR, β-arrestin1 or β-arrestin2 knockout mice exhibit significantly impaired responsiveness to ICL1-9 (FIGS. 8A & B). It's worth noting that ICL1-9-induced cardiomyocyte contraction was particularly dependent on β-arrestin1 (FIG. 8), perhaps reflecting the higher level of β-arrestin1 expression in the heart compared to β-arrestin2 (data not shown).

FIG. 8A depicts that β2AR-knockout, β-arrestin1-knockout and β-arrestin2-knockout adult murine cardiomyocytes were isolated and assessed for basal and agonist-promoted contractility using a digital video camera coupled microscope in the presence or absence of 0.1% DMSO or 10 μM ICL1-9. Representative cell length (in μm) tracings at 2 Hz in the basal or stimulated state for each test condition are reported. FIG. 8B depicts that ICL1-9 was unable to promote significant contraction in β2AR-knockout and β-arrestin1-knockout adult murine cardiomyocytes suggesting the mechanism of ICL1-9 contractility is dependent on the β2AR and β-arrestin1. The data are represented by the mean±SEM from 6-7 independent experiments. (ns—not significant, **—p<0.01, ***—p<0.001 using a one-way ANOVA with Newman-Keuls Multiple Comparison Test.).

ICL1-9 Decreases Infarct Size and Cell Death Following Ischemia-Reperfusion (I/R) Injury In Vivo.

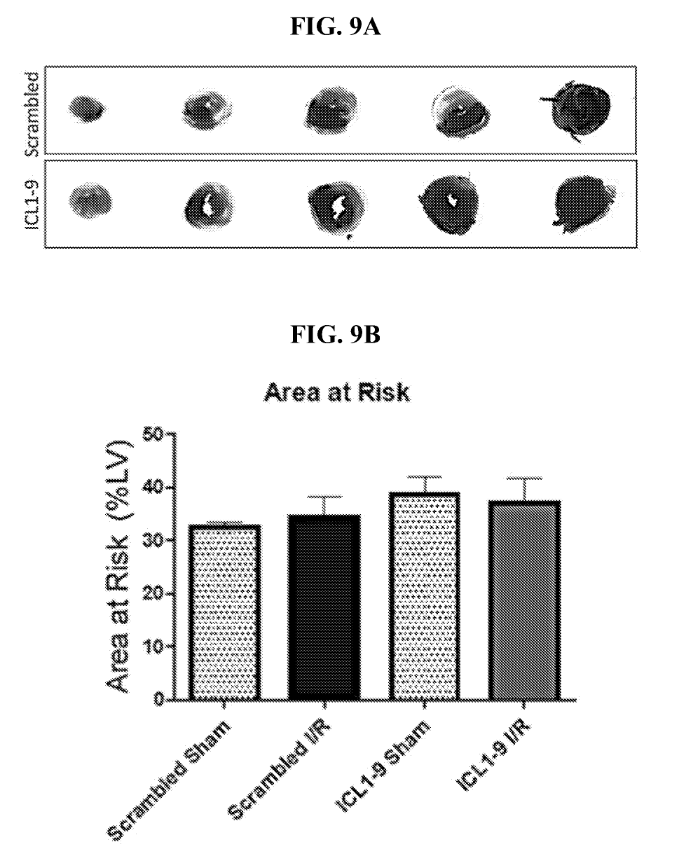

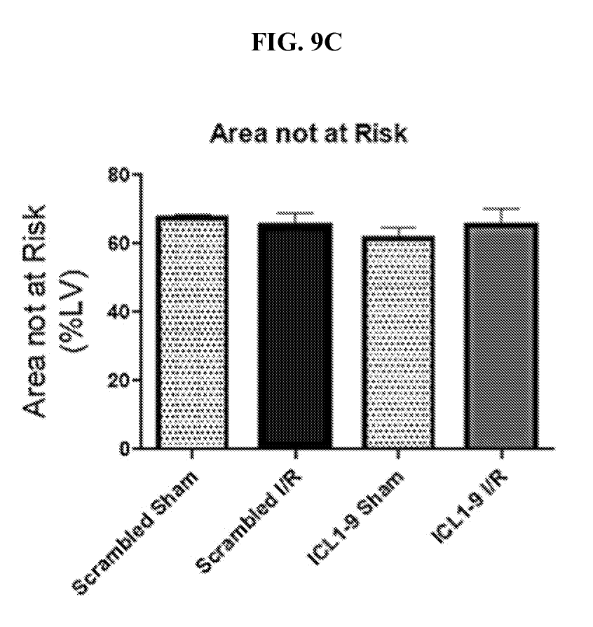

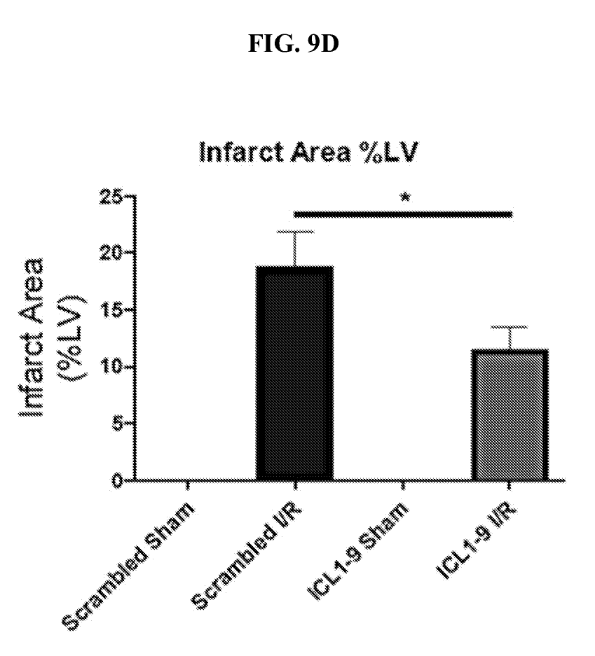

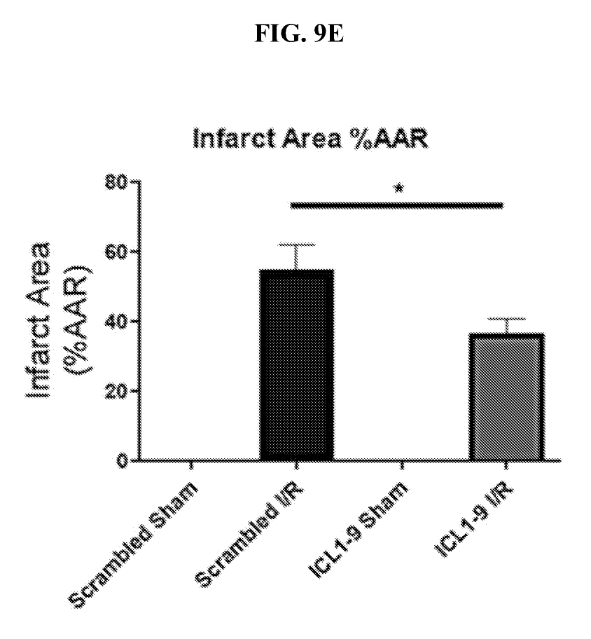

To evaluate the ability of ICL1-9 to function in vivo, ICL1-9 or scrambled pepducin were injected into the left ventricular wall of C57Bl/6 mice at three sites just prior to sham surgery or left descending coronary artery ligation for 60 min. After 24 hr of reperfusion, the hearts were perfused with Evans Blue dye and the mice were sacrificed, hearts frozen, stained with TTC, photographed (FIG. 9A) and risk and infarct areas calculated (FIG. 9B-E). While the area-at-risk (AAR) (FIG. 9B) and area-not-at-risk (FIG. 9C) were not significantly different between groups, the infarct area produced by FR injury, either as a percentage of total left ventricular (LV) area (FIG. 9D) or AAR (FIG. 9E), was significantly reduced in ICL1-9-treated mice. The data are the mean±SE, *p<0.05, One-way ANOVA, n=4 hearts for sham scrambled and sham ICL-9, n=8 for scrambled FR and n=11 for ICL1-9

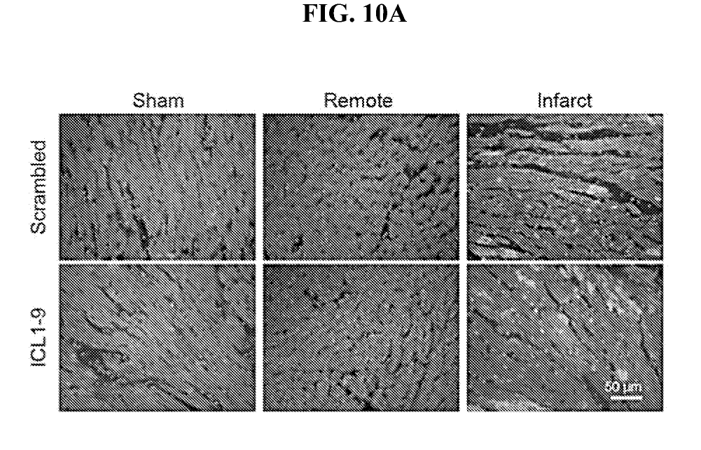

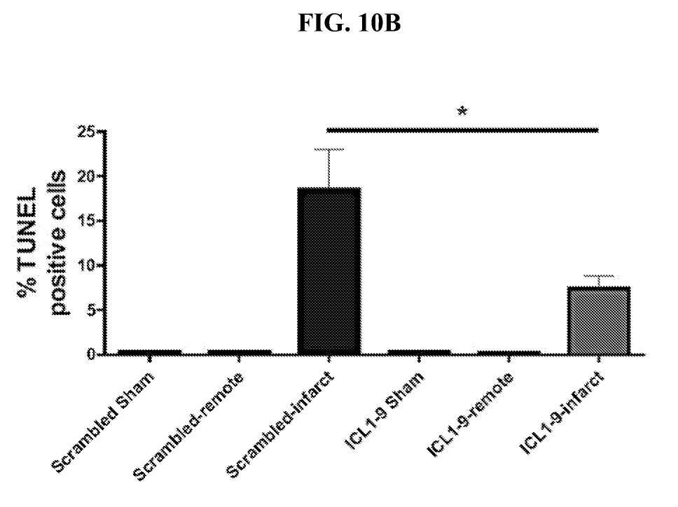

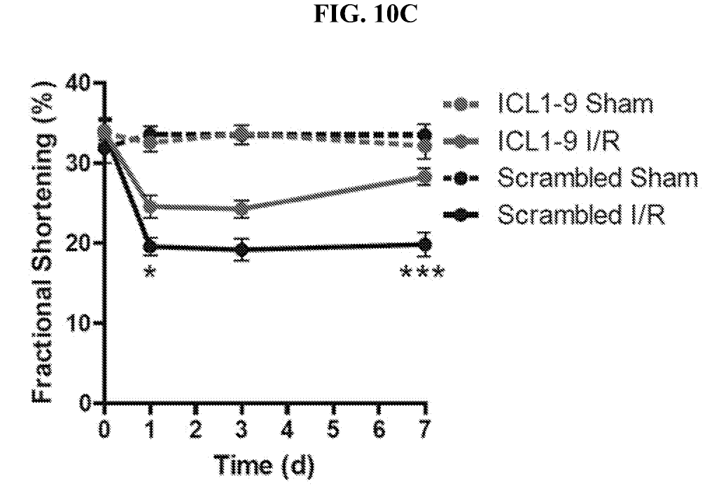

ICL1-9 was also assessed for its ability to protect cardiomyocytes from undergoing cell death in an ischemia-reperfusion (FR) injury model. Mice were treated and underwent surgical intervention as described above and after 24 hr reperfusion cell death staining (TUNEL, red), nuclear staining (DAPI, blue) and cardiomyocyte staining (α-sarcomeric actin, green) were performed on cardiac slices (FIG. 10A). Cell death within the infarct zone was significantly reduced by ICL1-9 versus scrambled pepducin (FIG. 10B). The data are mean±SE, *p<0.05, One-way ANOVA, n=4 hearts for sham scrambled and sham ICL-9, n=8 for scrambled FR and n=11 for ICL1-9 FR. As monitored via echocardiography, ICL1-9 significantly blunted FR-induced loss of cardiac contractility (FIG. 10C, % fractional shortening) compared to scrambled pepducin. The data are mean±SE, *p<0.05, ***p<0.001 scrambled FR vs ICL1-9 FR, Two-way repeated measures ANOVA, n=8 hearts for sham scrambled and sham ICL-9, n=12 for scrambled FR and ICL1-9

Discussion and Data Analysis

In the initial screens of the β2AR pepducin library, it was clear that β-arrestin-biased pharmacology was evidenced primarily in pepducins derived from ICL1 sequences (5). Further characterization revealed ICL1-9 as a potent (EC₅₀ of 96 nM) β-arrestin-biased pepducin that exhibits complete bias towards β-arrestin recruitment and signaling pathways over G protein activation. ICL1-9 promoted a pharmacological profile consistent with a β-arrestin-biased agonist, such as carvedilol, including receptor phosphorylation, internalization and β-arrestin-dependent signaling. Despite its similarity with carvedilol, ICL1-9 features three critical properties that carvedilol lacks. ICL1-9 operates independently of the orthosteric ligand binding pocket, it demonstrates specificity among β-adrenergic receptor family members and it can induce cardiomyocyte contraction. Conventional GPCR agonists, antagonists and inverse agonists operate through binding the receptor orthosteric ligand binding pocket and modulate the signaling propensity of the cognate receptor by influencing receptor conformational dynamics (agonist or inverse agonist) or simply competing for ligand binding (antagonist) (21). Carvedilol, for example, operates through interaction with the orthosteric ligand binding pocket (evidenced by [¹²⁵I]-iodocyanopindolol displacement) and is believed to stabilize a β-arrestin-biased receptor conformation that promotes β-arrestin-dependent processes and intracellular signaling (3). Similar analysis of ICL1-9 suggests that it operates independently of the orthosteric ligand-binding pocket to stimulate a signaling profile similar, yet not identical, to carvedilol. Although ICL1-9 does not influence receptor conformation in a conventional manner, it stabilizes a β₂AR conformation that is both a substrate for GRK-mediated phosphorylation and β-arrestin binding. By monitoring β₂AR-TM6 movement in vitro, ICL1-9 was observed to promote a significant conformational change in the β₂AR as well as interactions between the β₂AR and β-arrestin1 (WT and AAF). β-arrestin1-AAF is a mutant that lacks specific hydrophobic residues in the regulatory three-element region that creates a partially “pre-activated” form of β-arrestin1 that does not require the typical prerequisite GRK-mediated receptor phosphorylation in order to couple to the β₂AR (31). WT β-arrestin1 interaction, however, is enhanced by receptor phosphorylation and, thus, cannot couple to the β₂AR as efficiently in this assay. Finally, β-arrestin recruitment was sensitive to the inverse agonist ICI-118,551 which is believed to restrict receptor conformational dynamics. Although the particular conformational changes remain elusive, it is clear that ICL1-9 stabilizes a β-arrestin-biased β₂AR conformation independent of the orthosteric ligand binding site. This property provides the first opportunity to decouple β-arrestin-biased signaling from orthosteric site binding and may be a useful tool in studying the relative contribution of β-arrestin-dependent processes in the treatment of cardiovascular disease.

Pepducins are believed to operate through the cognate receptor in which they were derived although there is a growing body of evidence that their specificity, especially among closely related family members, must be considered (5, 6, 12-14). ICL1-9 demonstrated complete specificity towards the β₂AR compared to the β₁AR as ICL1-9 could not promote β-arrestin recruitment to the β₁AR or β₁AR internalization. Although both the β₁AR and the β₂AR are present in cardiomyocytes, in the normal heart, the β₁AR is the dominant subtype with an ˜4:1 expression ratio between the two subtypes (38). However, in the failing heart, the β₁AR is downregulated at the protein and mRNA level leading to a loss of ˜50% of the β₁AR while β₂AR expression remains unaltered (38, 39). Interestingly, each receptor subtype demonstrates distinct intracellular signaling pathways in the cardiomyocyte. Thus, in the failing heart, the changes in receptor subtype ratio can completely alter the intracellular signaling environment and regulatory crosstalk between the two pathways (33). As currently indicated CHF drugs are either β1AR-selective or non-selective agents, the β₂AR may be an underappreciated therapeutic target. β₁-selective pharmaceuticals, such as metoprolol and bisoprolol, are used in the treatment of CHF to inhibit the activation of cAMP-dependent, calmodulin-dependent kinase II (CaMKII) mediated apoptosis observed with persistent stimulation of the β₁AR (40). Activation of CaMKII, by complexing with β-arrestin1 and Epac, promoted agonist-dependent cardiac hypertrophy in vitro while also stimulating cardiac remodeling mechanisms in vivo (31, 32, 40, 41). This process is believed to be β1AR-specific and not mediated through the β₂AR (40). However, stimulation of the β₂AR during CHF may also contribute to the pathophysiological advancement of the syndrome. While β₁AR levels are reduced in the failing heart, Gᵢ, a heterotrimeric G protein that has been shown to couple to the β₂AR in the heart, is upregulated (42, 43). Gᵢ signaling reduces adenylyl cyclase activity and subsequent downstream inotropic responses critical for cardiac contraction while, in a Gβγ-dependent manner, promoting cell survival signaling such as Akt activation (44). The dichotomous nature of β₂AR signaling in the failing heart suggests that conventional receptor activation may not be the best therapeutic approach. In support of this concept, direct intramyocardial injection of ICL1-9 was able to reduce cardiomyocyte death, infarct size and cardiac contractile dysfunction following acute ischemia-reperfusion injury (FIGS. 9 and 10). Strikingly, these effects were produced by a single administration of ICL1-9 at the time of injury.

As conventional activation of β-adrenergic receptors is unlikely to be a viable therapeutic approach to treat CHF, a more desirable pharmacological profile would promote inotropic effects while also stimulating cell survival pathways. Indeed, a β-arrestin-biased agonist of the AT1R, TRV027, has been reported to promote cardiac contraction along with activation of anti-apoptotic signaling in cardiomyocytes and demonstrated promise through Phase 2b clinical trials for the treatment of heart failure (34-37). To date, a comparable ligand has not been reported for β-adrenergic receptors. As the inotropic effects of TRV027 are proposed to operate through a β-arrestin-mediated pathway, it is possible that a β-arrestin-biased βAR agonist, such as carvedilol, would be able to promote similar effects. Interestingly, carvedilol was unable to promote murine cardiomyocyte contraction while ICL1-9 promoted robust contraction. Carvedilol, biochemically characterized as a β-arrestin-biased agonist, can promote β-arrestin-mediated processes such as receptor internalization, ERK activation and EGFR transactivation but failed at promoting cardiac contraction. This may stem from an inability of carvedilol to stimulate an unknown β-arrestin dependent pathway through the β₂AR linked to contraction or, alternatively, the ability of carvedilol to interact with both β₁AR and β₂AR may have contrasting effects on myocyte contraction. In contrast, ICL1-9 was able to stimulate a β₂AR/β-arrestin complex that could both promote cell survival signaling pathways along with activation of cardiac inotropic effects. Although ICL1-9 does not promote cardiomyocyte contraction through traditional mechanisms (FIG. 7), it is possible that ICL1-9 couples β-arrestins to the myofilament proteins (as also proposed for TRV027). These mechanisms of calcium sensitization could operate through proteins that regulate the response to calcium such as myosin binding protein C or, more directly, troponin. Considering the ability to couple to the contractile machinery and pro-survival signaling pathways, ICL1-9 demonstrates that this potentially advantageous β-arrestin-biased conformation is accessible through the β₂AR and should be targeted for the next generation of heart failure therapeutics.

Therefore, the use of ICL1-9 pepducin in a pharmaceutical composition provides the following benefits to patients suffering an acute cardiac injury (i.e. myocardial infarction and/or ischemia/reperfusion) or in the context of either acute or chronic HF: (1) decreased catecholamine-induced Gs protein-dependent cardiotoxicity to decrease cell death; (2) active engagement of β-arrestin-dependent survival signaling to promote cell survival, thereby decreasing cell death-induced detrimental myocardial remodeling; and (3) increased cardiomyocyte contractility to actively improve cardiac function.

Because of the selective nature of the ICL1-9 and the ability of the pepducin to specifically bind β2AR without also inhibiting normal heart function, the pepducin can be used in combination with other treatments such as β1AR selective blockers.

It is further envisioned that ICL1-9 can be formulated into a pharmaceutical composition and administered to a patient by IV injection for non-specific administration. Alternatively, due to its hydrophobic properties, ICL1-9 can be packaged in a lipid bilayer delivery system (i.e. exosome or immunoliposome) for targeted delivery to the heart.

ICL1-9 can be administered by means that produces contact of the active agent with the agent's site of action. The ICL1-9 can be administered by conventional means available for use in conjunction with pharmaceuticals in a dosage range of 0.001 to 1000 mg/kg of mammal (e.g. human) body weight per day in a single dose or in divided doses. One dosage range is 0.01 to 500 mg/kg body weight per day in a single dose or in divided doses. Administration can be delivered as individual therapeutic agents or in a combination of therapeutic agents. They can be administered alone, but typically are administered with a pharmaceutically acceptable excipient selected on the basis of the chosen route of administration and standard pharmaceutical practice.

The ICL1-9 pepducin can be administered by one or more ways. For example, the following routes may be utilized: oral, parenteral (including subcutaneous injections, intravenous, intramuscular, intrasternal injection or infusion techniques), inhalation, buccal, sublingual, or rectal, in the form of a unit dosage of a pharmaceutical composition containing an effective amount of ICL1-9 and optionally in combination with one or more pharmaceutically-acceptable excipients such as stabilizers, anti-oxidants, lubricants, bulking agents, fillers, carriers, adjuvants, vehicles, diluents and other readily known excipients in standard pharmaceutical practice.

Liquid preparations suitable for oral administration (e.g. suspensions, syrups, elixirs and other similar liquids) can employ media such as water, glycols, oils, alcohols, and the like. Solid preparations suitable for oral administration (e.g. powders, pills, capsules and tablets) can employ solid excipients such as starches, sugars, kaolin, lubricants, binders, disintegrating agents, antioxidants and the like.

Parenteral compositions typically employ sterile water as a carrier and optionally other ingredients, such as solubility aids. Injectable solutions can be prepared, for example, using a carrier comprising a saline solution, a glucose solution or a solution containing a mixture of saline and glucose. Further guidance for methods suitable for use in preparing pharmaceutical compositions is provided in Remington: The Science and Practice of Pharmacy, 21st edition (Lippincott Williams & Wilkins, 2006).

Therapeutic compositions can be administered orally in a dosage range of about 0.001 to 1000 mg/kg of mammal (e.g. human) body weight per day in a single dose or in divided doses of the active ICL1-9 agent. One dosage range is about 0.01 to 500 mg/kg body weight per day orally in a single dose or in divided doses. For oral administration, the compositions can be provided in the form of tablets or capsules containing about 1 to 500 mg of the active ingredient, particularly about 1, 5, 10, 15, 20, 25, 50, 75, 100, 150, 200, 250, 300, 400, 500, and 750 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In view of the factors affecting the specific dose level and frequency it is contemplated that the dose frequency can range from multiple doses daily to monthly dosages. The preferred dose frequency ranges from twice a day to every two weeks. A more preferred dose frequency ranges from twice a day to weekly. A most preferred dose frequency ranges from twice a day to twice a week.

In the methods of various embodiments, pharmaceutical compositions including the active agent can be administered to a subject in an “effective amount.” An effective amount may be any amount that provides a beneficial effect to the patient, and in particular embodiments, the effective amount is an amount that may 1) prevent the subject from experiencing one or more adverse effects associated with administered agents, such as those used to diagnose, identify, and treat medical conditions, 2) reduce side effects experienced by the subject as a result of a medical therapy or reduce the side effects known to result from such therapies, and/or 3) eliminate side effects resulting from a medical treatment experienced by the subject prior to administration of the active agent or eliminate the side effects known to result from such treatment.

Pharmaceutical formulations containing ICL1-9 and a suitable carrier can be in various forms including, but not limited to, solids, solutions, powders, fluid emulsions, fluid suspensions, semi-solids, and dry powders including an effective amount of an the active agent of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's, The Pharmaceutical Basis of Therapeutics, 6th Edition, MacMillan Publishing Co., New York (1980) both of which are hereby incorporated by reference in their entireties can be consulted.

Further embodiments which may be useful for oral administration of the active agent include liquid dosage forms. In such embodiments, a liquid dosage may include a pharmaceutically acceptable emulsion, solution, suspension, syrup, and elixir containing inert diluents commonly used in the art, such as water. Such compositions may also comprise adjuvants, such as wetting agents, emulsifying and suspending agents, and sweetening, flavoring, and perfuming agents. Thus, for example, ICL1-9 can be formulated with suitable polymeric or hydrophobic materials (for example, as an emulsion in an acceptable oil) or ion exchange resins, or as sparingly soluble derivatives, for example, as a sparingly soluble salt. Other suitable diluents include, but are not limited to those described below:

Vegetable oil: As used herein, the term “vegetable oil” refers to a compound, or mixture of compounds, formed from ethoxylation of vegetable oil, wherein at least one chain of polyethylene glycol is covalently bound to the vegetable oil. In some embodiments, the fatty acids may have between about twelve carbons to about eighteen carbons. In some embodiments, the amount of ethoxylation can vary from about 2 to about 200, about 5 to 100, about 10 to about 80, about 20 to about 60, or about 12 to about 18 of ethylene glycol repeat units. The vegetable oil may be hydrogenated or unhydrogenated. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chemicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of Pharmaceutical Excipients, (2006), 5th ed., which is incorporated herein by reference in its entirety. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

Mineral oils: As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

Castor oils: As used herein, the term “castor oil,” refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-50 hydrogenated castor oil, and polyethylene glycol-60 hydrogenated castor oil, Emulphor™ EL-719 (castor oil 40 mole-ethoxylate, available from Stepan Products), the Cremophore™ series (available from BASF), which includes Cremophore RH40, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of Pharmaceutical Excipients, (2006), 5th ed., which is incorporated herein by reference in its entirety.

Sterol: As used herein, the term “sterol” refers to a compound, or mixture of compounds, derived from the ethoxylation of sterol molecule. Suitable polyethoyxlated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko).

Polyethylene glycol: As used herein, the term “polyethylene glycol” or “PEG” refers to a polymer containing ethylene glycol monomer units of formula —O—CH2-CH2-. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

Propylene glycol fatty acid ester: As used herein, the term “propylene glycol fatty acid ester” refers to a monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

Stearoyl macrogol glyceride: Stearoyl macrogol glyceride refers to a polyglycolized glyceride synthesized predominately from stearic acid or from compounds derived predominately from stearic acid, although other fatty acids or compounds derived from other fatty acids may be used in the synthesis as well. Suitable stearoyl macrogol glycerides include, but are not limited to, Gelucire® 50/13 (available from Gattefosse).

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

Exemplary excipients or carriers for use in solid and/or liquid dosage forms include, but are not limited to:

Sorbitol: Suitable sorbitols include, but are not limited to, PharmSorbidex E420 (available from Cargill), Liponic 70-NC and 76-NC (available from Lipo Chemical), Neosorb (available from Roquette), Partech SI (available from Merck), and Sorbogem (available from SPI Polyols).

Starch, sodium starch glycolate, and pregelatinized starch include, but are not limited to, those described in R. C. Rowe and P. J. Shesky, Handbook of Pharmaceutical Excipients, (2006), 5th ed., which is incorporated herein by reference in its entirety.

Disintegrant: The disintegrant may include one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

Still further embodiments of the invention include the active agent administered in combination with other active such as, for example, adjuvants, protease inhibitors, or other compatible drugs or compounds where such combination is seen to be desirable or advantageous in achieving the desired effects of the methods described herein.

Other embodiments of the present invention include a pharmaceutical composition comprising an effective amount of the active agent and one or more pharmaceutically acceptable excipient. In yet other embodiments, the active agent may be combined with one or more secondary agents.

Further embodiments would allow deliver of ICL1-9 through a viral mediated delivery system or perhaps via direct injection into the heart. Viral mediated delivery systems are known to one of ordinary skill in the art. The delivery system for the ICL1-9 pepducin can include either a viral or non-viral vector delivery vehicles. Viral gene delivery systems include recombinant virus vectors such as adenovirus vectors, retrovirus vectors, pox-virus vectors, mutant viruses (described above) and virosomes. Non-viral gene delivery systems include DNA conjugates with sugar, polylysine, polyethylenimine, polyethylenimine derivatives, and liposomes, together with their derivatives.

Non-viral gene delivery systems such as those utilizing sugars, sugar derivatives, liposomes, liposome derivatives and polyethylenimine or polyethylenimine derivatives can be utilized in certain embodiments.

### REFERENCES

1. Javed U & Deedwania P C (2009) Beta-adrenergic blockers for chronic heart failure. Cardiol Rev 17(6):287-292.
2. Ramani G V, Uber P A, & Mehra M R (2010) Chronic heart failure: contemporary diagnosis and management. Mayo Clin Proc 85(2):180-195.
3. Wisler J W, et al. (2007) A unique mechanism of beta-blocker action: carvedilol stimulates beta-arrestin signaling. Proc Natl Acad Sci USA 104(42):16657-16662.
4. Erickson C E, et al. (2013) The beta-blocker Nebivolol Is a GRK/beta-arrestin biased agonist. PLoS One 8(8):e71980.
5. Carr R, 3rd, et al. (2014) Development and characterization of pepducins as Gs-biased allosteric agonists. J Biol Chem 289(52):35668-35684.
6. O'Callaghan K, Kuliopulos A, & Covic L (2012) Turning receptors on and off with intracellular pepducins: new insights into G-protein-coupled receptor drug development. J Biol Chem 287(16):12787-12796.
7. Lefkowitz R J (2007) Seven transmembrane receptors: something old, something new. Acta Physiol (Oxf) 190(1):9-19.
8. Tran T M, et al. (2004) Characterization of agonist stimulation of cAMP-dependent protein kinase and G protein-coupled receptor kinase phosphorylation of the beta2-adrenergic receptor using phosphoserine-specific antibodies. Mol Pharmacol 65(1):196-206.
9. Vaughan D J, et al. (2006) Role of the G protein-coupled receptor kinase site serine cluster in beta2-adrenergic receptor internalization, desensitization, and beta-arrestin translocation. J Biol Chem 281(11):7684-7692.
10. Nobles K N, et al. (2011) Distinct phosphorylation sites on the beta(2)-adrenergic receptor establish a barcode that encodes differential functions of beta-arrestin. Sci Signal 4(185):ra51.
11. Kang D S, Tian X, & Benovic J L (2014) Role of beta-arrestins and arrestin domain-containing proteins in G protein-coupled receptor trafficking. Curr Opin Cell Biol 27:63-71.
12. Covic L, Misra M, Badar J, Singh C, & Kuliopulos A (2002) Pepducin-based intervention of thrombin-receptor signaling and systemic platelet activation. Nat Med 8(10):1161-1165.
13. Stampfuss J J, Schror K, & Weber A A (2003) Inhibition of platelet thromboxane receptor function by a thrombin receptor-targeted pepducin. Nat Med 9(12):1447; author reply 1447-1448.
14. Winther M, Gabl M, Welin A, Dahlgren C, & Forsman H (2015) A neutrophil inhibitory pepducin derived from FPR1 expected to target FPR1 signaling hijacks the closely related FPR2 instead. FEBS Lett.
15. Quoyer J, et al. (2013) Pepducin targeting the C-X-C chemokine receptor type 4 acts as a biased agonist favoring activation of the inhibitory G protein. Proc Natl Acad Sci USA 110(52):E5088-5097.
16. DeWire S M, Ahn S, Lefkowitz R J, & Shenoy S K (2007) Beta-arrestins and cell signaling. Annu Rev Physiol 69:483-510.
17. Morrison D K & Davis R J (2003) Regulation of MAP kinase signaling modules by scaffold proteins in mammals. Annu Rev Cell Dev Biol 19:91-118.
18. Patel P A, Tilley D G, & Rockman H A (2008) Beta-arrestin-mediated signaling in the heart. Circ J 72(11):1725-1729.
19. Noma T, et al. (2007) Beta-arrestin-mediated beta1-adrenergic receptor transactivation of the EGFR confers cardioprotection. J Clin Invest 117(9):2445-2458.
20. Kim I M, et al. (2008) Beta-blockers alprenolol and carvedilol stimulate beta-arrestin-mediated EGFR transactivation. Proc Natl Acad Sci USA 105(38):14555-14560.
21. Luttrell L M (2014) Minireview: More than just a hammer: ligand “bias” and pharmaceutical discovery. Mol Endocrinol 28(3):281-294.
22. Chidiac P, Hebert T E, Valiquette M, Dennis M, & Bouvier M (1994) Inverse agonist activity of beta-adrenergic antagonists. Mol Pharmacol 45(3):490-499.
23. Samama P, Pei G, Costa T, Cotecchia S, & Lefkowitz R J (1994) Negative antagonists promote an inactive conformation of the beta 2-adrenergic receptor. Mol Pharmacol 45(3):390-394.
24. Szczepek M, et al. (2014) Crystal structure of a common GPCR-binding interface for G protein and arrestin. Nat Commun 5:4801.
25. Shukla A K, et al. (2014) Visualization of arrestin recruitment by a G-protein-coupled receptor. Nature 512(7513):218-222.
26. Rasmussen S G, et al. (2011) Crystal structure of the beta2 adrenergic receptor-Gs protein complex. Nature 477(7366):549-555.
27. Yao X, et al. (2006) Coupling ligand structure to specific conformational switches in the beta2-adrenoceptor. Nat Chem Biol 2(8):417-422.
28. Yao X J, et al. (2009) The effect of ligand efficacy on the formation and stability of a GPCR-G protein complex. Proc Natl Acad Sci USA 106(23):9501-9506.
29. Burtey A, et al. (2007) The conserved isoleucine-valine-phenylalanine motif couples activation state and endocytic functions of beta-arrestins. Traffic 8(7):914-931.
30. Zhu W Z, et al. (2003) Linkage of beta1-adrenergic stimulation to apoptotic heart cell death through protein kinase A-independent activation of Ca2+/calmodulin kinase II. J Clin Invest 111(5):617-625.
31. Bisognano J D, et al. (2000) Myocardial-directed overexpression of the human beta(1)-adrenergic receptor in transgenic mice. J Mol Cell Cardiol 32(5):817-830.
32. Engelhardt S, Hein L, Wiesmann F, & Lohse M J (1999) Progressive hypertrophy and heart failure in beta1-adrenergic receptor transgenic mice. Proc Natl Acad Sci USA 96(12):7059-7064.
33. Woo A Y & Xiao R P (2012) beta-Adrenergic receptor subtype signaling in heart: from bench to bedside. Acta Pharmacol Sin 33(3):335-341.
34. Violin J D, et al. (2010) Selectively engaging beta-arrestins at the angiotensin II type 1 receptor reduces blood pressure and increases cardiac performance. J Pharmacol Exp Ther 335(3):572-579.
35. Boerrigter G, et al. (2011) Cardiorenal actions of TRV120027, a novel □-arrestin-biased ligand at the angiotensin II type I receptor, in healthy and heart failure canines: a novel therapeutic strategy for acute heart failure. Circ Heart Fail 4(6):770-778.
36. Felker G M, et al. (2015) Heart failure therapeutics on the basis of a biased ligand of the angiotensin-2 type 1 receptor. Rationale and design of the BLAST-AHF study (Biased Ligand of the Angiotensin Receptor Study in Acute Heart Failure). JACC Heart Fail 3(3):193-201.
37. Soergel D G, Subach R A, Cowan C L, Violin J D, & Lark M W (2013) First clinical experience with TRV027: pharmacokinetics and pharmacodynamics in healthy volunteers. J Clin Pharmacol 53(9):892-899.
38. Bristow M R, et al. (1986) Beta 1- and beta 2-adrenergic-receptor subpopulations in nonfailing and failing human ventricular myocardium: coupling of both receptor subtypes to muscle contraction and selective beta 1-receptor down-regulation in heart failure. Circ Res 59(3):297-309.
39. Bristow M R, et al. (1993) Reduced beta 1 receptor messenger RNA abundance in the failing human heart. J Clin Invest 92(6):2737-2745.
40. Mangmool S, Shukla A K, & Rockman H A (2010) beta-Arrestin-dependent activation of Ca(2+)/calmodulin kinase II after beta(1)-adrenergic receptor stimulation. J Cell Biol 189(3):573-587.
41. Sucharov C C, et al. (2006) A beta1-adrenergic receptor CaM kinase II-dependent pathway mediates cardiac myocyte fetal gene induction. Am J Physiol Heart Circ Physiol 291(3):H1299-1308.
42. Bohm M, et al. (1994) Radioimmunochemical quantification of Gi alpha in right and left ventricles from patients with ischaemic and dilated cardiomyopathy and predominant left ventricular failure. J Mol Cell Cardiol 26(2):133-149.
43. Feldman A M, et al. (1988) Increase of the 40,000-mol wt pertussis toxin substrate (G protein) in the failing human heart. J Clin Invest 82(1):189-197.
44. Zhu W Z, et al. (2001) Dual modulation of cell survival and cell death by beta(2)-adrenergic signaling in adult mouse cardiac myocytes. Proc Natl Acad Sci USA 98(4):1607-1612.
45. Daunt D A, et al. (1997) Subtype-specific intracellular trafficking of alpha2-adrenergic receptors. Mol Pharmacol 51(5): 711-720.
46. Rosenbaum D M, et al. (2007) GPCR engineering yields high-resolution structural insights into beta2-adrenergic receptor function. Science 318(5854):1266-1273.
47. Wang J, et al. (2011) Regulation of in vivo cardiac contractility by phospholemman: role of Na+/Ca2+ exchange. Am J Physiol Heart Circ Physiol 300(3):H859-868.

What is claimed is:

1. (canceled)

2. (canceled)

3. (canceled)

4. A method of treating cardiovascular diseases by administering to a patient a pharmaceutical composition comprising a pepducin, said pepducing having a binding affinity for β2AR.

5. The method of claim 4, wherein the pharmaceutical composition stimulates cardioprotective signaling and inotropic effects through the β2AR.

6. The method of claim 4, wherein the pharmaceutical composition induces cardiomyocyte contraction.

7. The method of claim 4, wherein the pharmaceutical composition enhances contractile function.

8. A pharmaceutical composition for treating cardiovascular diseases comprising a pepducin having SEQ ID No. 1, wherein said pharmaceutical composition operates independently of the orthosteric ligand binding pocket to stimulate a signaling pathway that promotes contraction of the heart, and wherein said pharmaceutical composition stabilizes a β2AR conformation that is both a substrate for GRK-mediated phosphorylation and β-arrestin binding.

9. The method of claim 4, for treating cardiovascular diseases wherein, the pharmaceutical composition is an ICL1-9 pepducin in a pharmaceutical composition; wherein said administration to a patient (1) decreased catecholamine-induced Gs protein-dependent cardiotoxicity to decrease cell death; (2) active engagement of βarrestin-dependent survival signaling to promote cell survival, thereby decreasing cell death-induced detrimental myocardial remodeling; and (3) increased cardiomyocyte contractility to actively improve cardiac function.

10. The method of claim 9 wherein the pharmaceutical composition can be formulated to be administered to a patient by IV injection for non-specific administration.

11. The method of claim 9 wherein the pharmaceutical composition can be packaged in a lipid bilayer delivery system (i.e. exosome or immunoliposome) for targeted delivery to the heart.

12. The method of claim 9 wherein the pharmaceutical composition can be packaged in a viral delivery system for targeted delivery to the heart.

13. A pharmaceutical composition comprising ICL1-9 have a sequence SEQ ID No. 1, wherein said composition stimulates cardiomyocyte contractility and activating the β2AR/β-arrestin signaling pathway.

14. The composition of claim 13 which further simultaneously prevents cardiotoxic G protein-dependent βAR signaling.

15. The composition of claim 13 which further promotes pro-survival signaling of cardiomyocyte cells.

16. (canceled)

17. (canceled)

18. (canceled)

19. (canceled)

20. A formulation comprising an active agent selected from the group consisting of ICL1-4, ICL1-11, ICL1-20, or ICL1-9, or combinations thereof for the treatment of cardiovascular disease, heart disease, myocardial infarction, ischemia, reperfusion injury, AHF, CHF, and other acute and chronic diseases.

21. The formulation of claim 20 wherein the active agent includes a sequence of ICL1-4, ICL1-11, ICL1-20, or ICL1-9 having 90% homology to each of these pepducins.

22. The formulation of claim 20 further comprising a specific β1AR inhibitor such as bisoprolol or metoprolol.

23. The formulation of claim 1, wherein said active agent is an ICL1-9 pepducin having SEQ ID No. 1, for use in the treatment of cardiovascular disease, suitable for administration to a patient in an effective dose for treatment of the cardiovascular disease.

24. (canceled)

25. The formulation of claim 20, wherein the pharmaceutical composition comprising pepducin ICL1-9 (SEQ ID No. 1), SEQ ID No. 2, SEQ ID No. 3, SEQ ID No. 4, or combinations thereof suitable for treatment of one or more of heart disease, myocardial infarction, ischemia, reperfusion injury, AHF, CHF, and other acute and chronic diseases.

26. (canceled)

27. (canceled)

28. The method of claim 4, wherein said pepducin is selected from the group consisting of ICL1-4, ICL1-11, ICL1-20, or ICL1-9, or combinations thereof.

29. The method of claim 4, wherein the pepducin comprises SEQ ID No. 1, which binds to β2AR and stimulates cardiac contractility.

30. The method of claim 4, wherein the pepducin comprises SEQ ID No. 1, which binds to β2AR and stimulates cardiac contractility and prevent myocardial remodeling.

31. The method of claim 4, wherein the pepducin comprises SEQ ID No. 1, which binds to β2AR, stimulates cardiomyocyte function, and promotes cardiomyocyte survival and contractility.

32. The method of claim 4, wherein the pepducin comprises the sequence consisting of pepducin ICL1-9 (SEQ ID No. 1), SEQ ID No. 2, SEQ ID No. 3, or SEQ ID No. 4.
